(19) United States
(12) Patent Application Publication
(10) Pub. No.: US 2025/0346611 A1
(43) Pub. Date: Nov. 13, 2025

(54) FLUORESCENT GTP ANALOGUES AND USE

(71) Applicant: CISBIO BIOASSAYS, Codolet (FR)

(72) Inventors: Laurent LAMARQUE, SAINT-VICTOR LA COSTE (FR); Emmanuel BOURRIER, BAGNOLS-SUR-CEZE (FR); Thomas ROUX, NIMES (FR); Eric TRINQUET, PONT-SAINT-ESPRIT (FR); Elodie DUPUIS, CAISSARGUES (FR)

(21) Appl. No.: 19/273,225

(22) Filed: Jul. 18, 2025

Related U.S. Application Data
(63) Continuation of application No. 17/427,200, filed on Jul. 30, 2021, now Pat. No. 12,398,158.

(30) Foreign Application Priority Data
Jan. 30, 2019 (FR) 1900856

(51) Int. Cl. C07F 5/00

(52) U.S. Cl. CPC C07F 5/00

(57) ABSTRACT

The invention relates to compounds of formula:

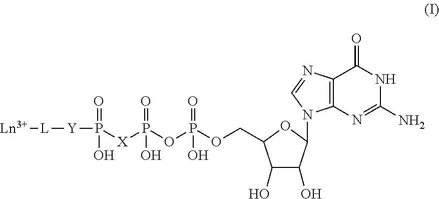

in which X, Y, L and Ln³⁺ are as defined in the description.

### CROSS REFERENCE TO RELATED APPLICATIONS

This application is a continuation of U.S. application Ser. No. 17/427,200, which is the U.S.

national stage entry of PCT/FR2020/050149, filed on Jan. 30, 2020, which claims priority to and all benefit of French Patent Application No. 1900856, filed on Jan. 30, 2019, the entire disclosures of which are fully incorporated herein by reference.

### FIELD OF THE INVENTION

The present invention relates to fluorescent GTP analogs useful for detecting, by energy transfer techniques, molecules capable of modulating the activation of a G protein-coupled receptor.

### STATE OF THE ART

G protein-coupled receptors (GPCRs) are a family of membrane receptors in mammals and throughout the animal kingdom. G proteins are heterotrimeric proteins (3 subunits: alpha, beta and gamma) which are activated by GPCRs. Through the GPCRs, the G proteins have a role of transducing a signal from outside the cell to the inside of the cell (i.e. cellular response to an external stimulus). Their commonly described mechanism of action is presented in FIG. 1 and summarized below:
in its inactive, resting, state, the alpha subunit of the G protein is bound to the nucleotide GDP (full GDP-bound G protein);
after activation of the GPCR, the latter binds to the alpha subunit of the G protein and initiates a process of activation of the G protein consisting of two stages:
1) the departure of the GDP from the G protein to give an empty G protein, and the formation of an inactive GPCR/empty G protein complex, and
2) the binding of GTP which results in the formation of an active G protein, in GTP form (full GTP-bound G protein). In the first stage, the G protein bound to the receptor is in a form called “empty form”. This state is described in the literature as being transient since it is described that the nucleotide GTP binds rapidly to the alpha subunit of the G protein. In addition, the beta/gamma subunits of the activated G protein dissociate from the alpha subunit;
the alpha subunit of the full GTP-bound G protein then binds to the effectors in order to activate them. The effectors in their turn activate signaling pathways resulting in a cellular response;
the GTP is subsequently hydrolyzed to give GDP by the alpha subunit of the G protein and the alpha subunit reassociates with the beta/gamma subunits to reform the full GDP-bound G protein (inactive state).

There exist several subtypes of G alpha proteins exhibiting different selectivity profiles for the different effectors (Journal of Molecular Biology, 2016, 428, 3850) and thus bringing about the activation of preferential signaling pathways.

GPCRs are associated with many important physiological functions and are considered to be one of the favored therapeutic targets for a large number of pathologies. Thus, many in vitro screening tests have been developed in order to identify molecules capable of modulating GPCRs. The tests developed make use of different mechanisms for the activation of G proteins and employ varied technologies (Zhang et al.; Tools for GPCR Drug Discovery; Acta Pharmacologica Sinica, 2012, 33, 372). Mention may in particular be made of affinity tests which use radiolabeled ligands to measure the affinity of the ligand for GPCR, proximity scintigraphy tests which use scintigraphy beads to which GPCRs have been attached or functional tests using weakly or non-hydrolyzable GTP, such as GTPγS (GTP-gamma-S). These tests are nevertheless difficult to carry out and sometimes require membrane filtration stages which can limit their use as high-throughput screening (HTS) tests. Other tests have been developed to demonstrate the activation of GPCRs. These tests are based in particular on energy transfer techniques (RET—Resonance Energy Transfer), such as FRET (Fluorescence Resonance Energy Transfer)—see Clinical Chemistry, 1995, 41, 1391—or BRET (Bioluminescence Resonance Energy Transfer)—see Proceedings of the National Academy of Sciences, 1999, 96(1), 151. These two techniques involve notions of molecules capable of giving energy (referred to as donors) or of accepting energy (referred to as acceptors)—see Physical Chemistry Chemical Physics, 2007, 9, 5847. Mention may be made, for example, of the energy transfer techniques demonstrating the interaction between a GPCR and the G protein by using either a donor conjugated to the GPCR and an acceptor conjugated to the G protein (WO 2006/086883 and WO 2003/008435) or an acceptor conjugated to the alpha subunit of the G protein and a donor conjugated to the beta and/or gamma subunit of the G protein (Bunemann et al., Proceedings of the National Academy of Sciences, 2003, 26, 16077). These techniques are nevertheless restrictive since they require the preparation of fusion proteins and they do not make it possible to study the GPCRs and the G proteins expressed endogenously by the cells (i.e. unmodified and not overexpressed). On the other hand, in order to discriminate between the different subtypes of G alpha proteins which can be activated by the receptor, these techniques require the preparation of multiple membrane samples (a specific preparation for each subtype of G alpha protein). Energy transfer techniques have also been used for the development of tests targeted at visualizing the modulation of the GTP (active) form of the G protein or of the GDP (inactive) form of the G protein. Mention may be made, for example, of the applications WO 2006/035208 and US 2007/0287162, in which a GTP analog coupled to a cyanine-type molecule is employed.

Application WO 2009/068751 describes a method in which an energy transfer signal is detected using labeled ATP derivatives; these derivatives cannot, however, bind to the G protein. A GTP analog is described in the journal Drug Discovery Today, 2002, 7(18), S150), as capable of being used in a time-resolved fluorescence detection technique. This analog consists of a europium chelate coupled to the phosphate atom in the gamma position of GTP via a nitrogen atom. The structure of the europium chelate, however, is not disclosed, no more than the method used to synthesize the analog in question. Another GTP analog is described in Analytical Chemistry, 2009, 81, 5033, which results from the coupling of gamma-[(8-aminooctyl)imido]guanosine-5′-triphosphate and {2,2′,2″,2′″-{[[2-(4-isothiocyanatophenyl)ethyl]imino]bis(methylene)bis{4-{[4-(R-D-glucopyranoxy)phenyl]ethynyl}pyridine-6,2-diyl}bis(methylenenitrilo)}tetrakis(acetato)}europium(III), the synthesis of which has been described in Analytical Chemistry, 2003, 75, 3193.

There thus exists a real need to have available compounds capable of binding to the G protein and which can de facto be used in a method for the detection, by energy transfer techniques, of molecules capable of modulating the activation of a G protein-coupled receptor.

### SUMMARY OF THE INVENTION

An object of the present invention is the provision of new molecules of GTP or derivatives thereof, which are coupled to lanthanide complexes and which are represented by the general formula (I):

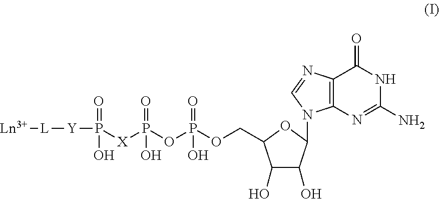

in which:
X=O, NH or CH₂;
Y=O, NH or CH₂;
L is a divalent linking group;
Ln³⁺ is a lanthanide complex which can optionally carry a reactive group G₃ (as defined below);
it being understood that, when X represents NH and L-Y represents an octylamino group, then Ln³⁺ is not {2,2′,2″,2″′-{[[2-(4-isothiocyanatophenyl)ethyl]imino]bis(methylene)bis{4-{[4-(R-D-glucopyranoxy)phenyl]ethynyl}pyridine- 6,2-diyl}bis(methylenenitrilo)}tetrakis (acetato)}europium(III).

### BRIEF DESCRIPTION OF THE DRAWINGS

FIG. 1 illustrates the mechanism of action of G proteins.

FIG. 2 illustrates the assay format used to detect the binding of the compounds of the invention to the G protein.

FIG. 3 illustrates a test for binding a compound of the invention to the Gαi protein.

FIG. 4 illustrates a test for binding a compound of the invention to the Gαi protein.

FIG. 5 illustrates a test for binding a compound of the invention to the Gαi protein.

FIG. 6 illustrates a test for binding a compound of the invention to the Gαi protein.

FIG. 7 illustrates a test for binding a compound of the invention to the Gαi protein.

FIG. 8 illustrates a test for binding a compound of the invention to the Gαi protein.

FIG. 9 illustrates a test for binding a compound of the invention to the Gαi protein.

FIG. 10 illustrates a test for binding a compound of the invention to the Gαi protein.

FIGS. 11A and 11B illustrate the effect of the concentration of membranes and of test compound on the binding of said compound to the Gαi protein.

FIG. 12 illustrates a test for binding a compound of the invention to the Gαi protein.

### DESCRIPTION OF THE INVENTION

According to one aspect, the present invention relates to compounds of formula (I):

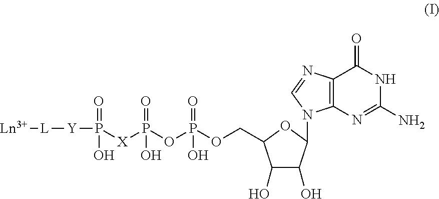

in which:
X=O, NH or CH₂;
Y=O, NH or CH₂;
L is a divalent linking group;
Ln³⁺ is a lanthanide complex which can optionally carry a reactive group G₃ (as defined below);
provided that, when X represents NH and L-Y represents an octylamino group, then Ln³⁺ is not {2,2′,2″,2″′-{[[2-(4-isothiocyanatophenyl)ethyl]imino]bis(methylene)bis{4-{[4-(R-D-glucopyranoxy)phenyl]ethynyl}pyridine-6,2-diyl}bis(methylenenitrilo)}tetrakis (acetato)}europium(III).

The term “lanthanide complex” is understood to mean a chelate, a macrocycle, a cryptate or any organic entity capable of complexing an atom of the lanthanide family, the lanthanide (Ln) being chosen from: Eu, Sm, Tb, Gd, Dy, Nd or Er; preferably, the lanthanide is Tb, Sm or Eu and more preferably still Eu or Tb.

The families of the various compounds of formula (I) are represented by the formulae (Ia) to (Ig):

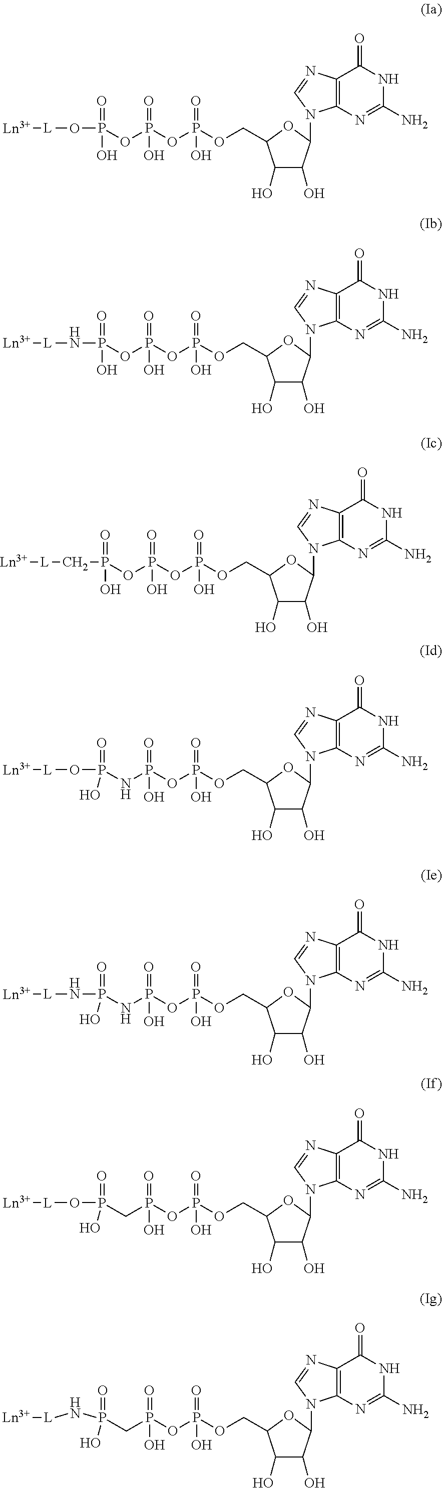

in which L and Ln³⁺ have the meanings indicated above.

A first family of compounds according to the invention consists of the compounds of formulae (Ia), (Id) and (If). This family is called the GTP-gamma-O family (because the divalent linking group is bonded to the phosphate in the gamma position of the GTP via an oxygen atom). A second family of compounds according to the invention consists of the compounds of formulae (Ib), (Ie) and (Ig). This family is called the GTP-gamma-N family (because the divalent linking group is bonded to the phosphate in the gamma position of the GTP via a nitrogen atom). A third family of compounds according to the invention consists of the compounds of formula (Ic). This family is called the GTP-gamma-C family (because the divalent linking group is bonded to the phosphate in the gamma position of the GTP via a carbon atom).

In one embodiment, X is O. In another embodiment, X is NH. In another embodiment, X is CH₂.

In one embodiment, the divalent linking group L is chosen from:
a direct link;
a linear or branched C₁-C₂₀, preferably C₁-C₈, alkylene group, optionally containing one or more double or triple bonds;
a C₅-C₈ cycloalkylene group; or
a C₆-C₁₄ arylene group;
said alkylene, cycloalkylene or arylene groups optionally containing one or more heteroatoms, such as oxygen, nitrogen, sulfur or phosphorus, or one or more carbamoyl or carboxamido group(s), and said alkylene, cycloalkylene or arylene groups optionally being substituted by 1 to 5, preferably 1 to 3, C₁-C₈ alkyl, C₆-C₁₄ aryl, sulfonate or oxo groups.

The divalent linking group L is advantageously chosen from the following groups:

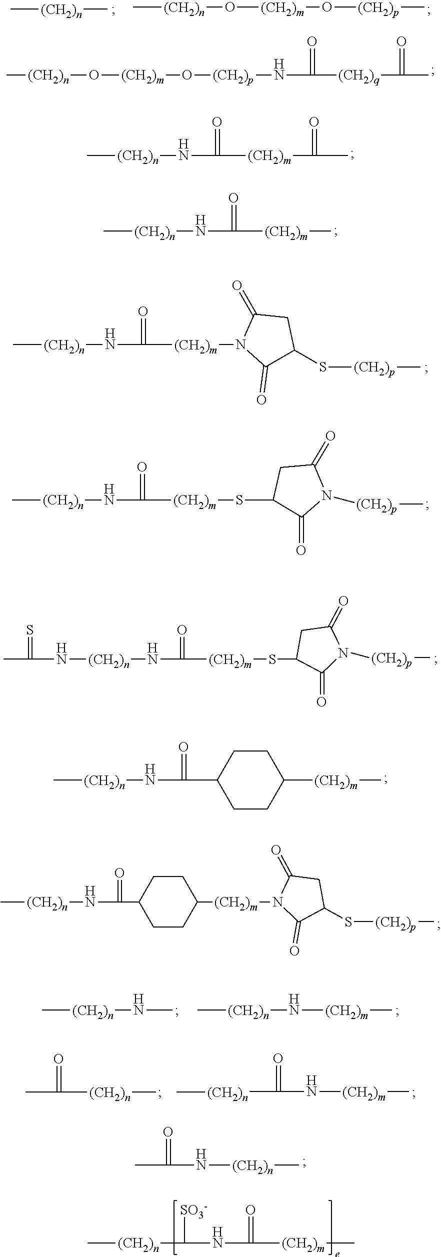

in which n, m, p and q are integers from 1 to 16, preferably from 1 to 5, and e is an integer ranging from 1 to 6, preferably from 1 to 4.

In a particularly advantageous way, the divalent linking group L is chosen from a direct link, a linear or branched C₁-C₈ alkylene group or a group of formula:

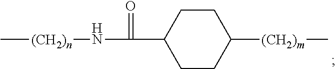

The divalent linking group L is preferably chosen from:

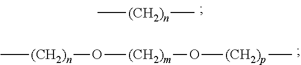

the —(CH₂)ₙ- group being very particularly preferred.

According to another embodiment, the divalent linking group L is a group of formula:

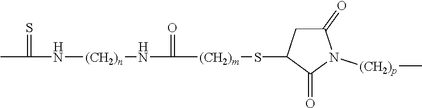

in which m, n and p are integers from 1 to 16, preferably from 1 to 5.

In one embodiment, the lanthanide complex Ln³⁺ is chosen from one of the complexes below:

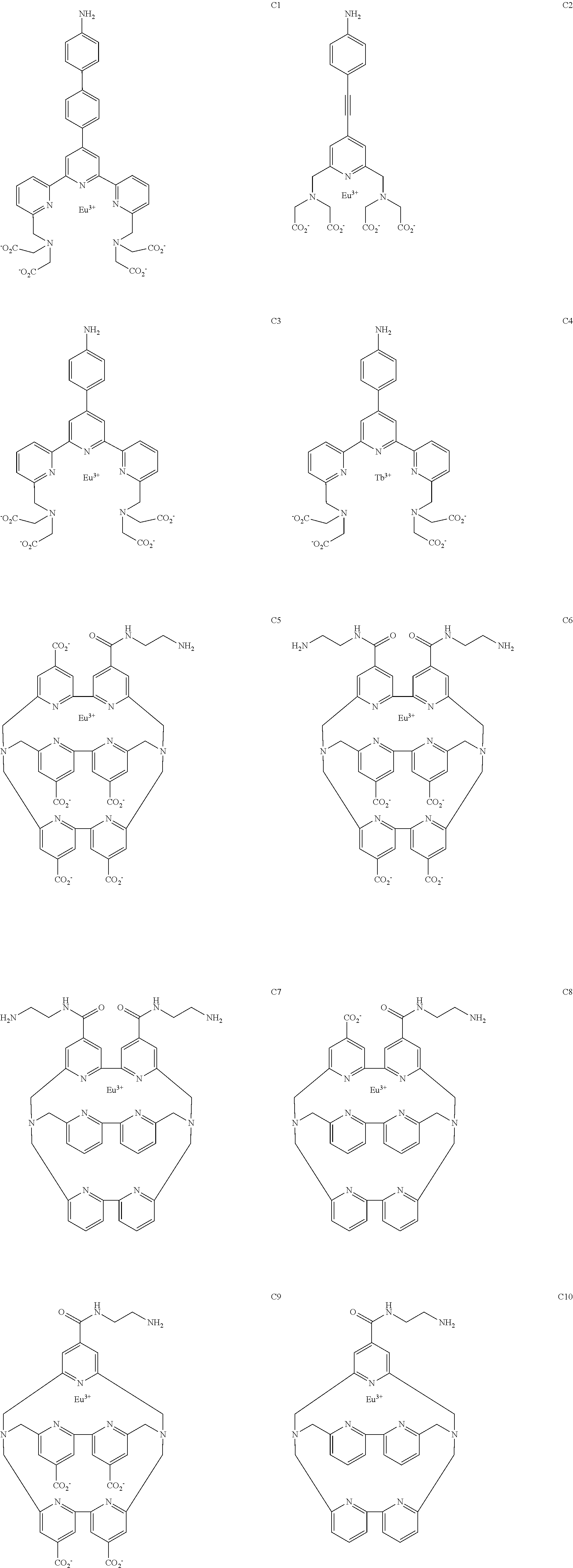

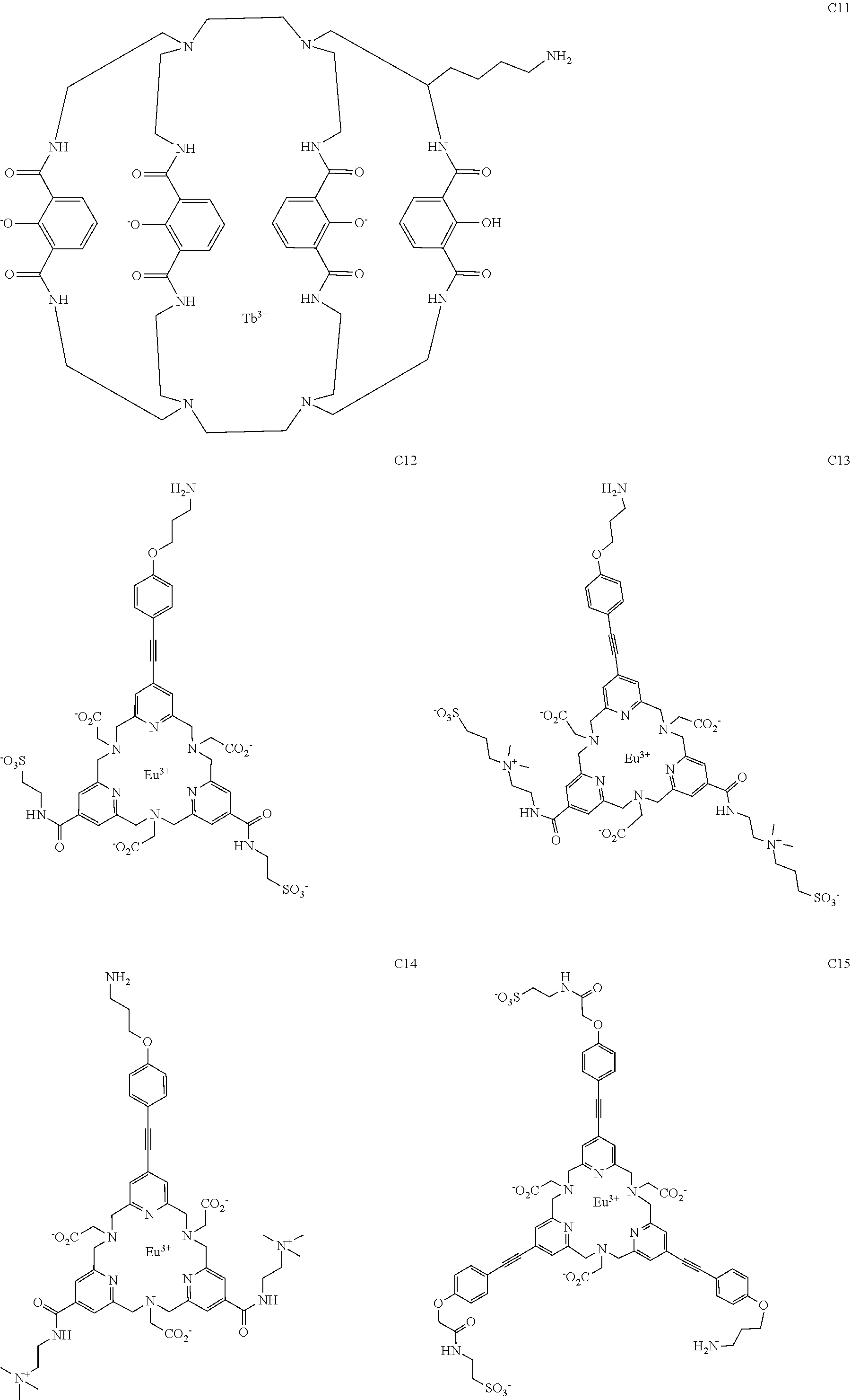

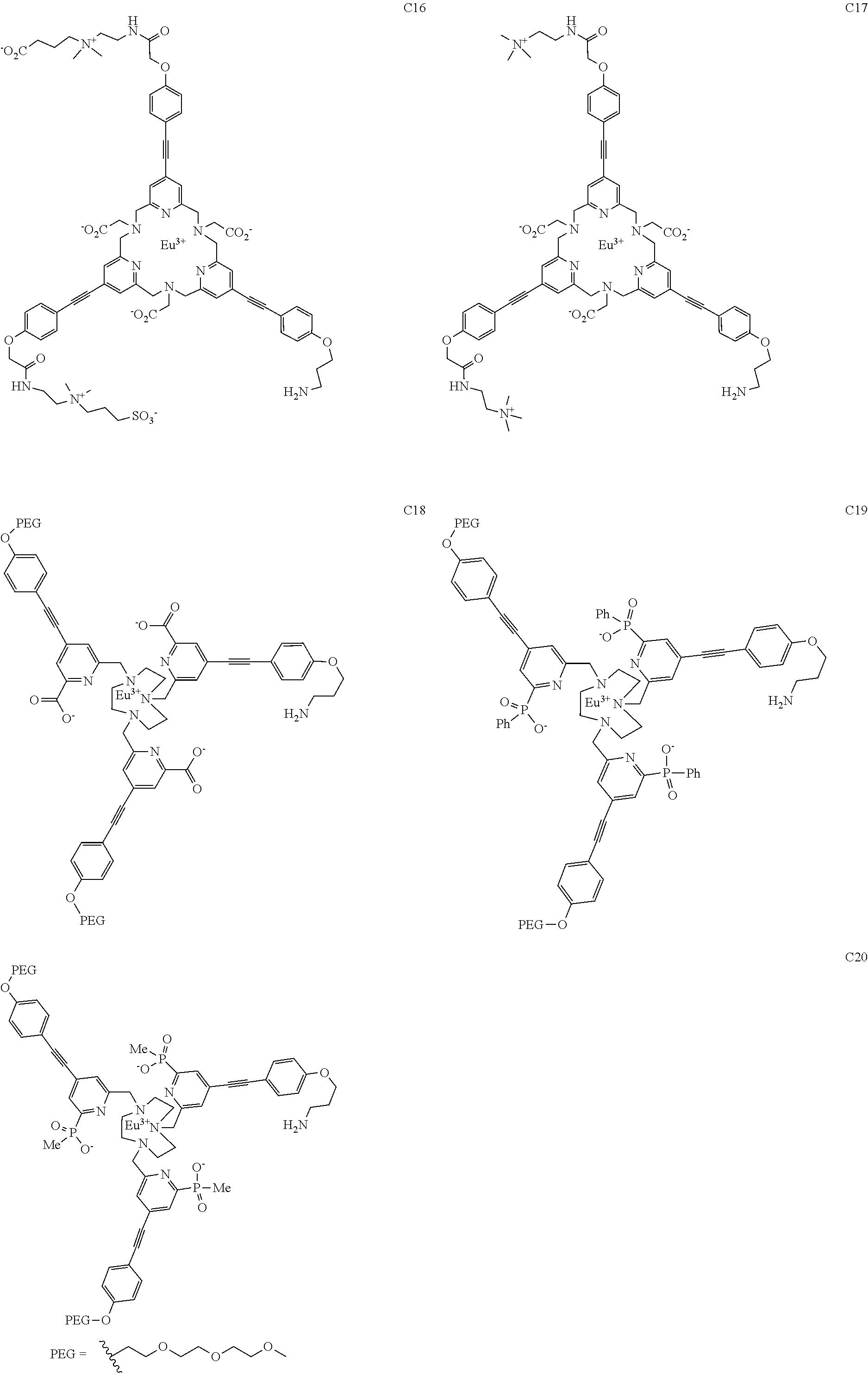

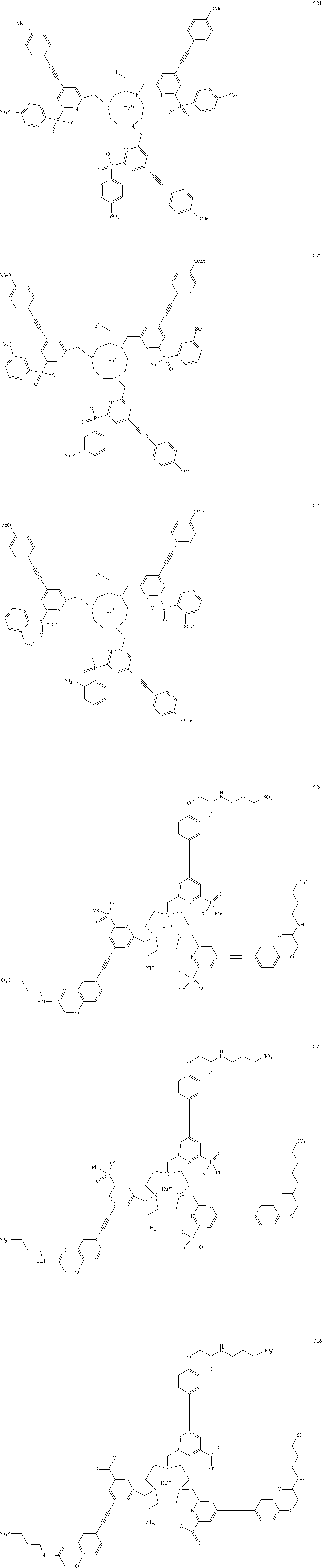

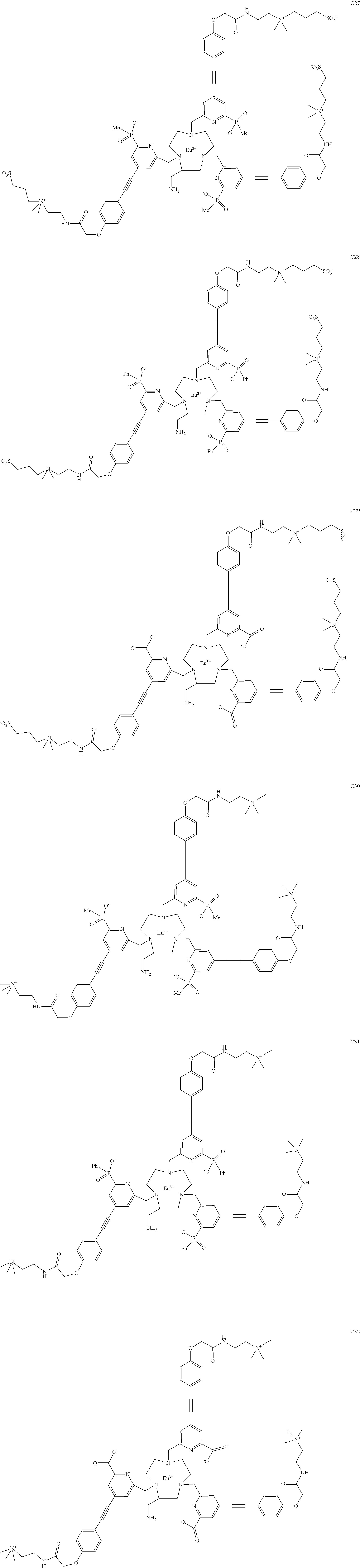

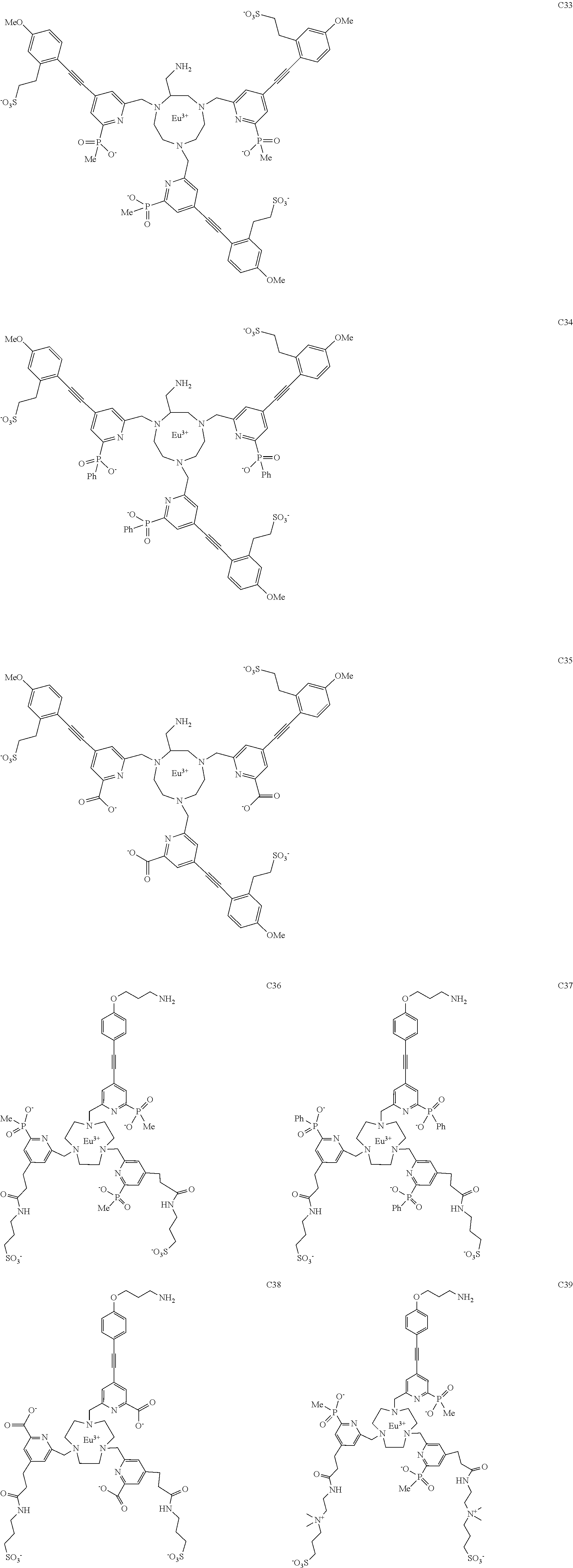

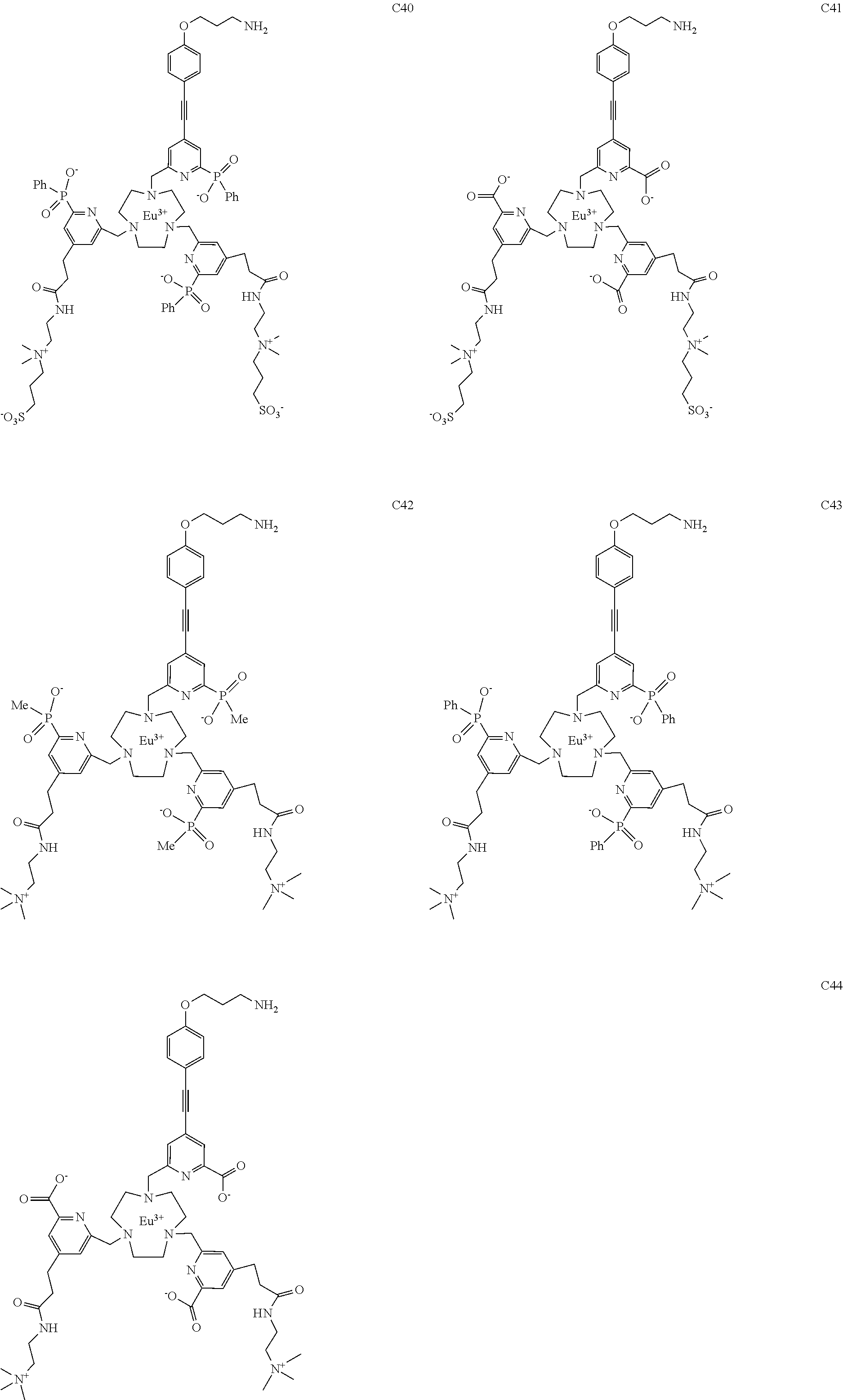

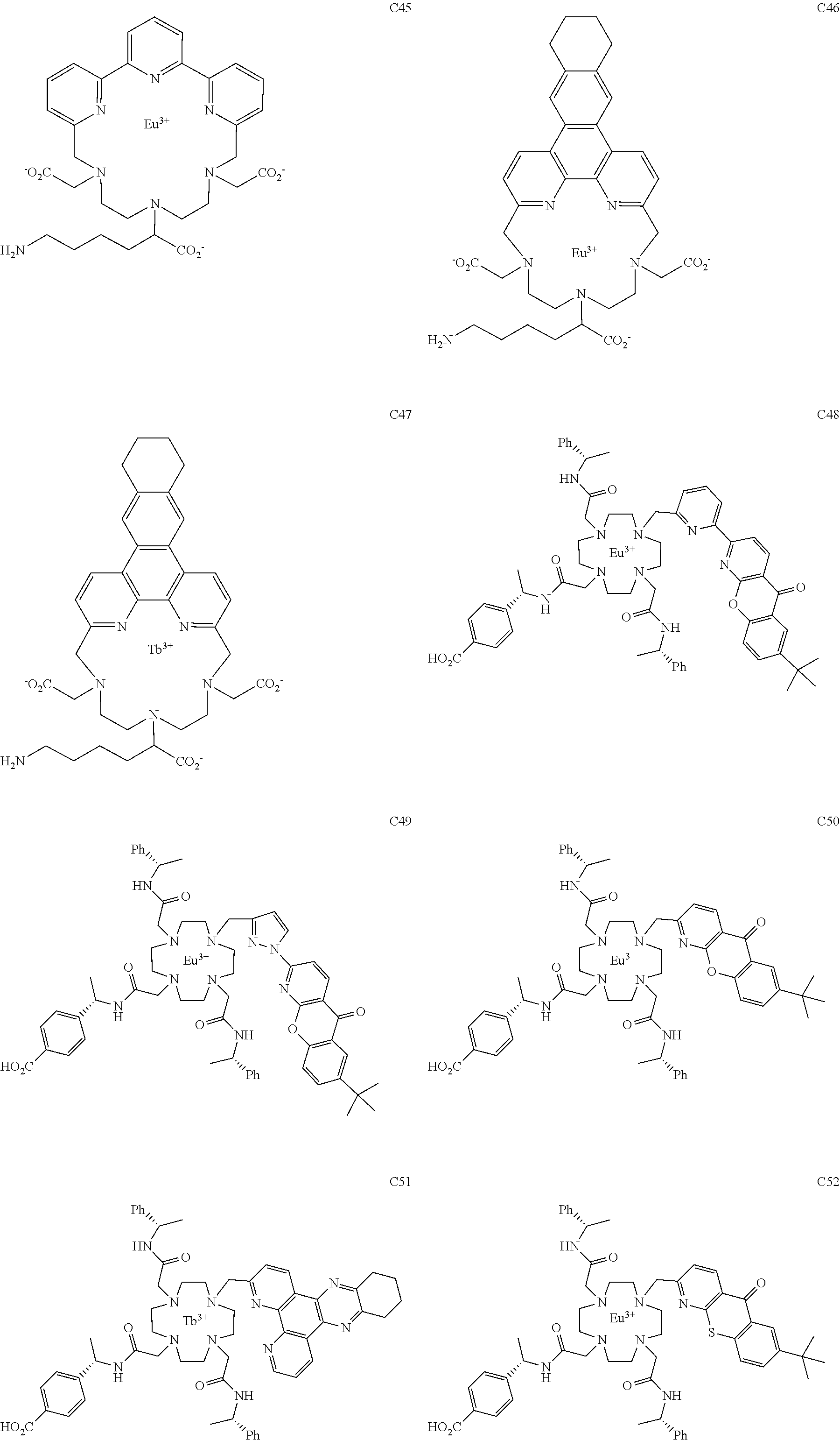

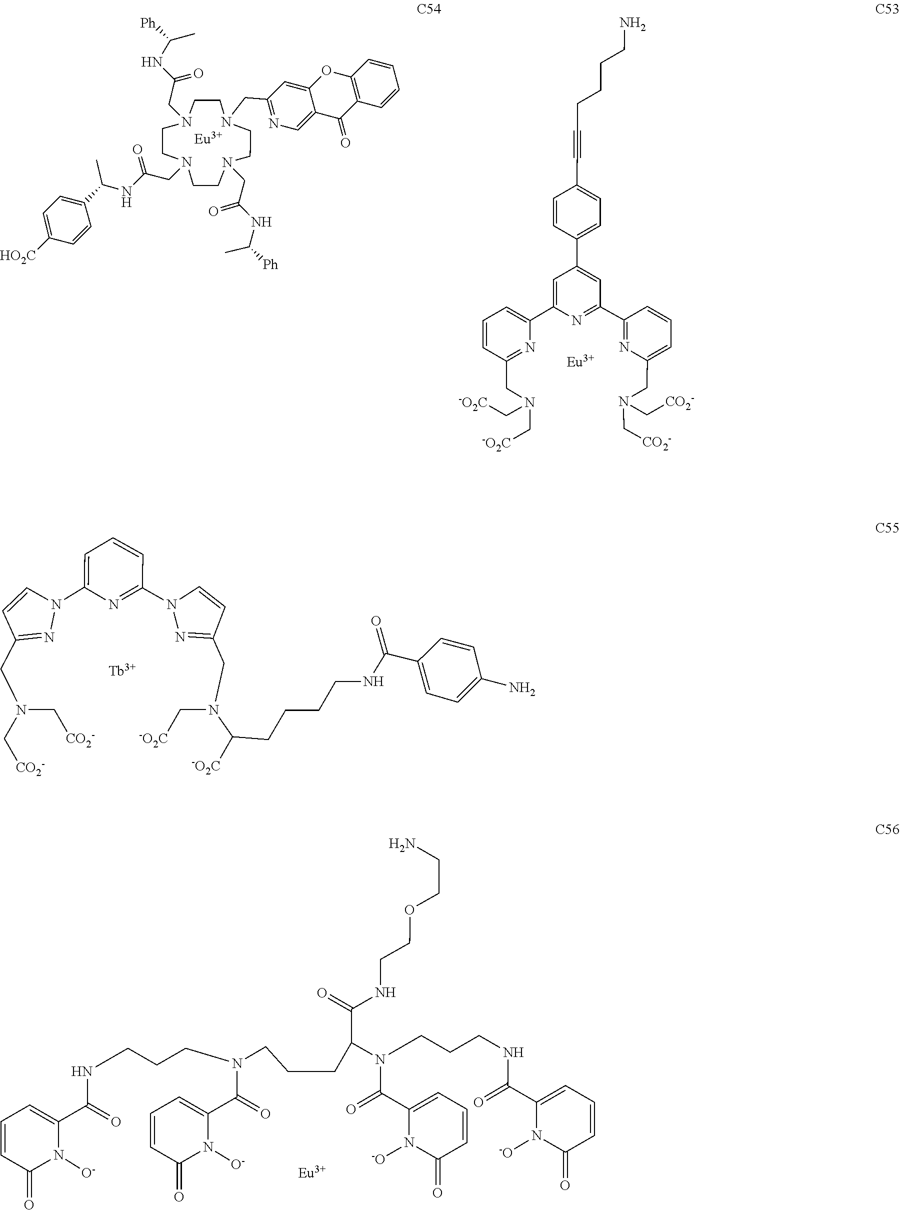

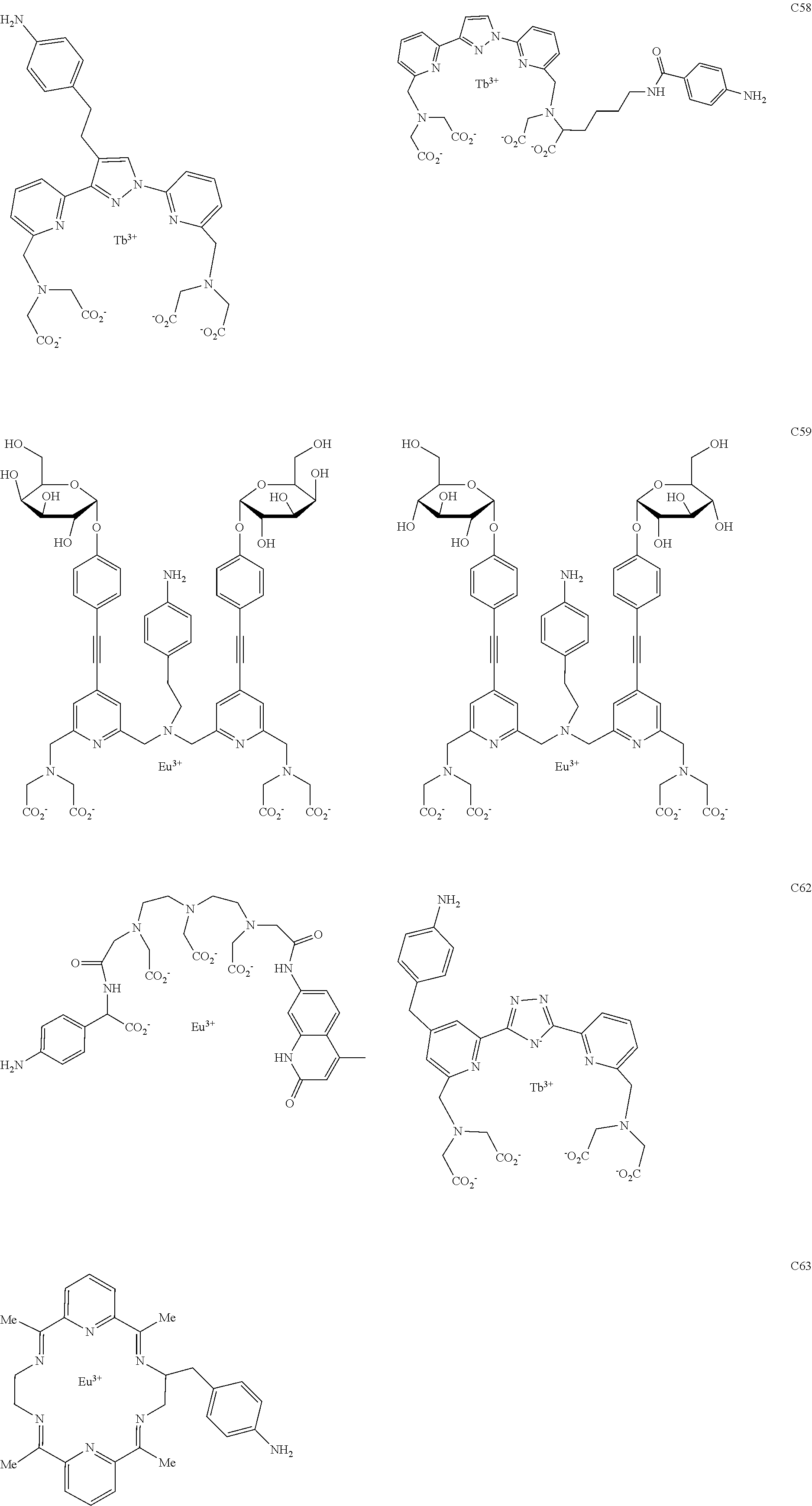

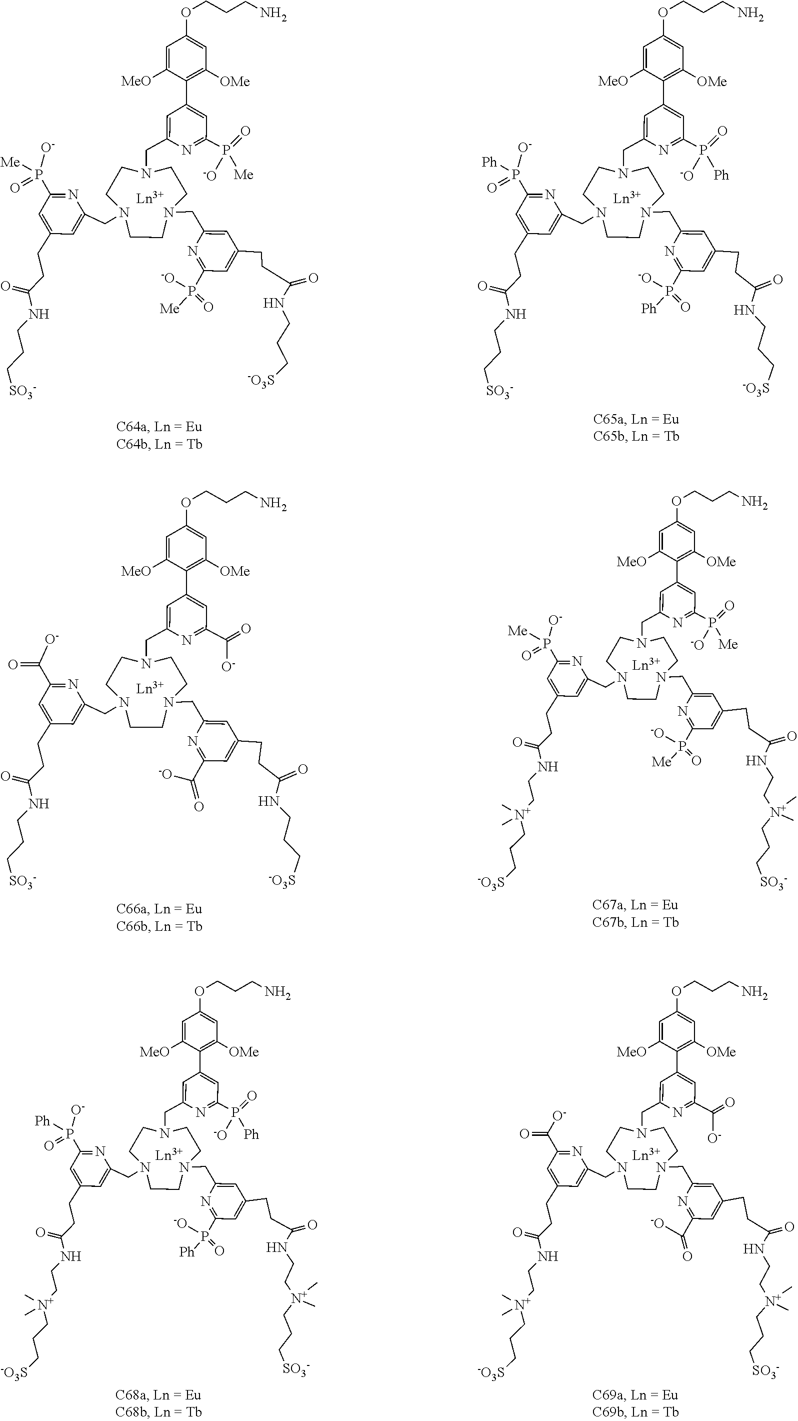

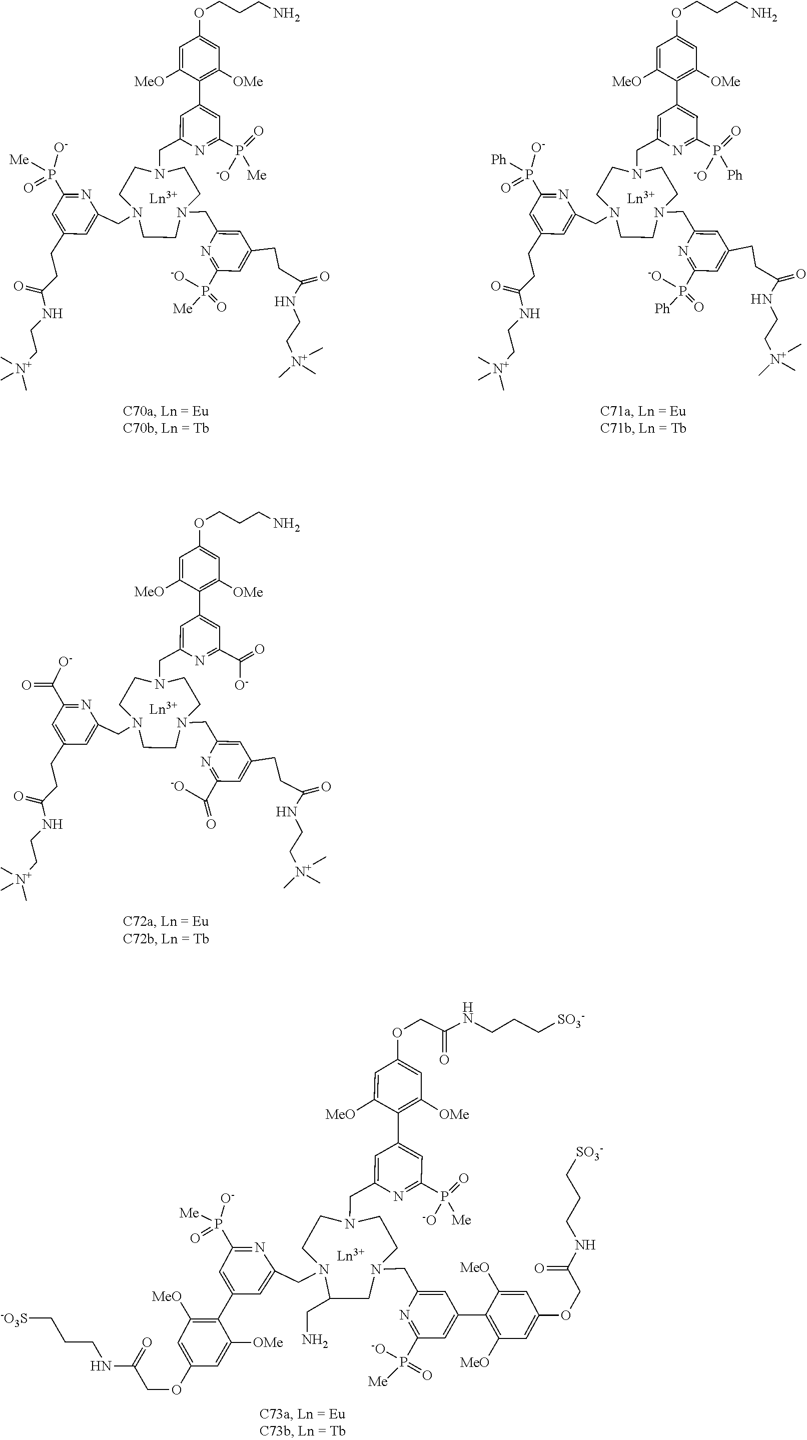

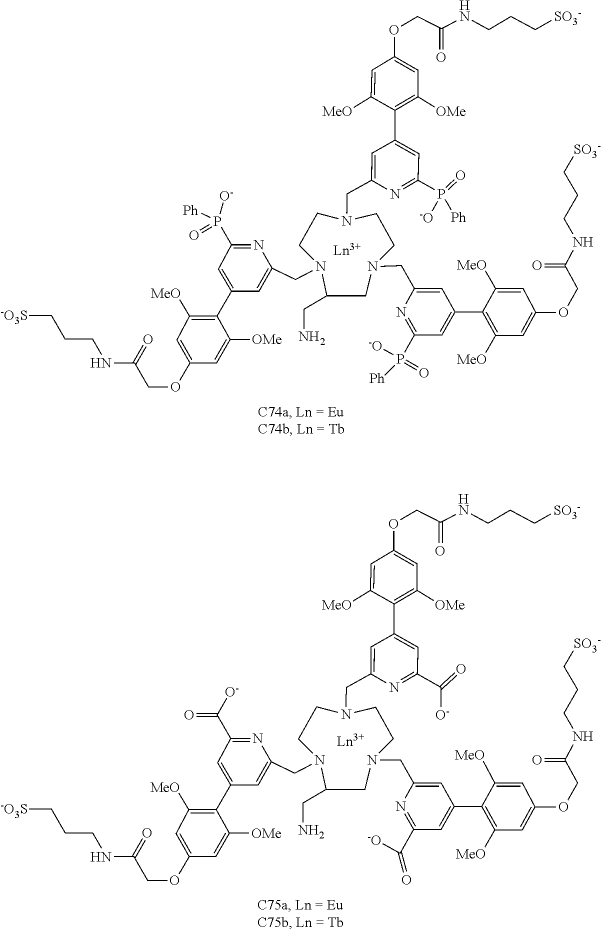

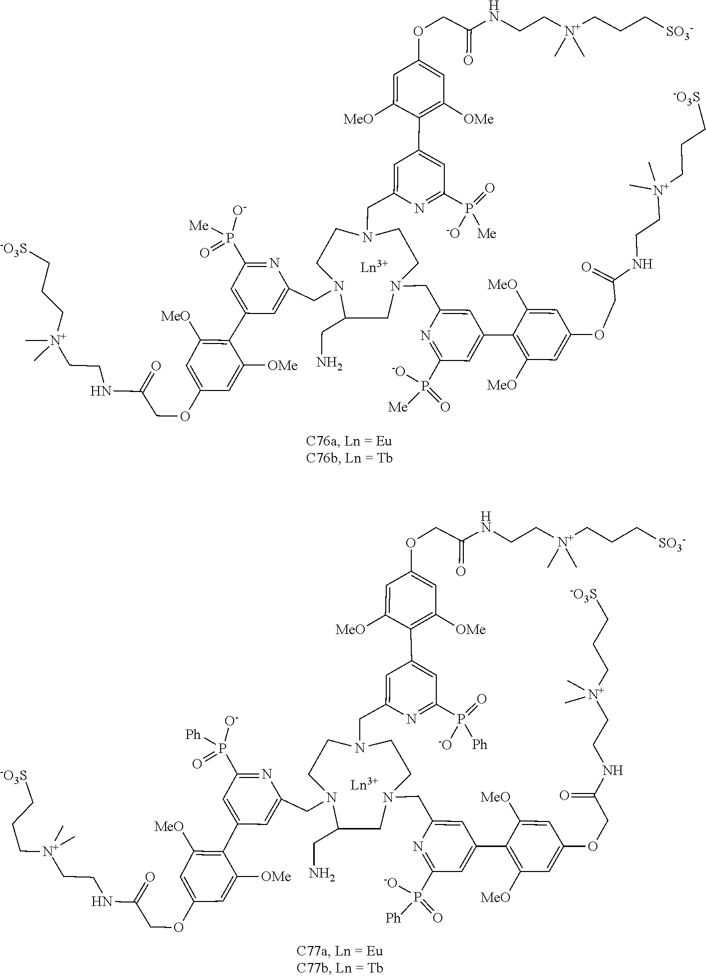

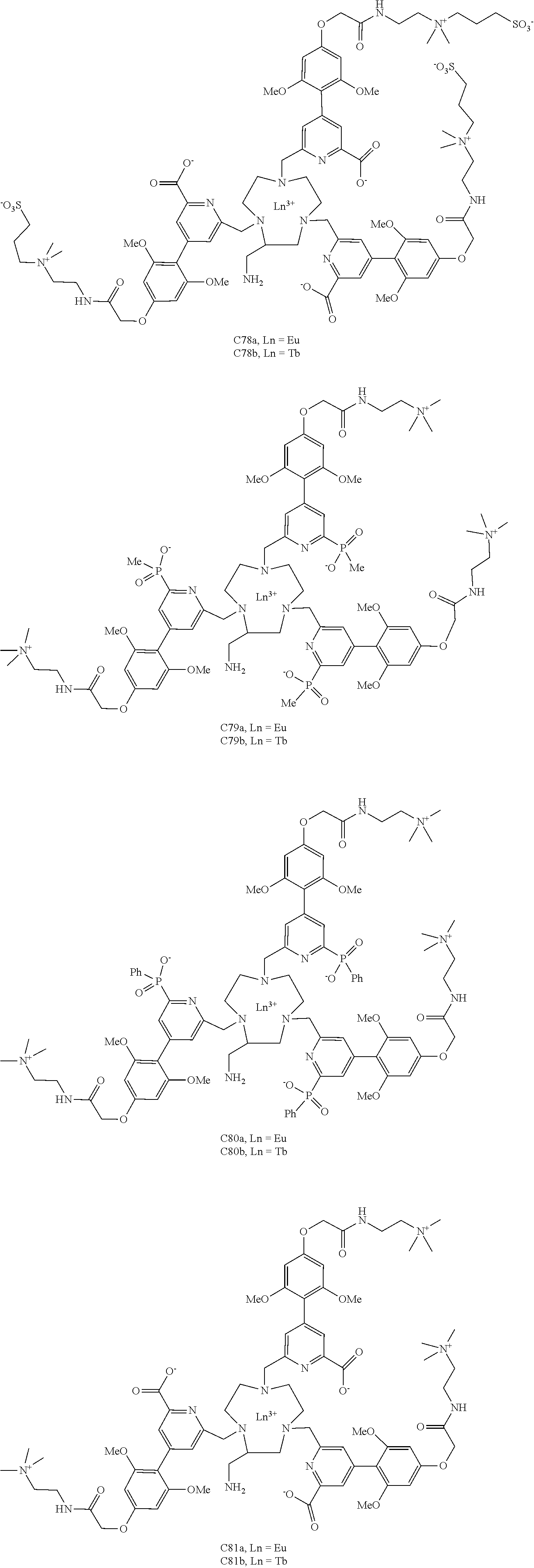

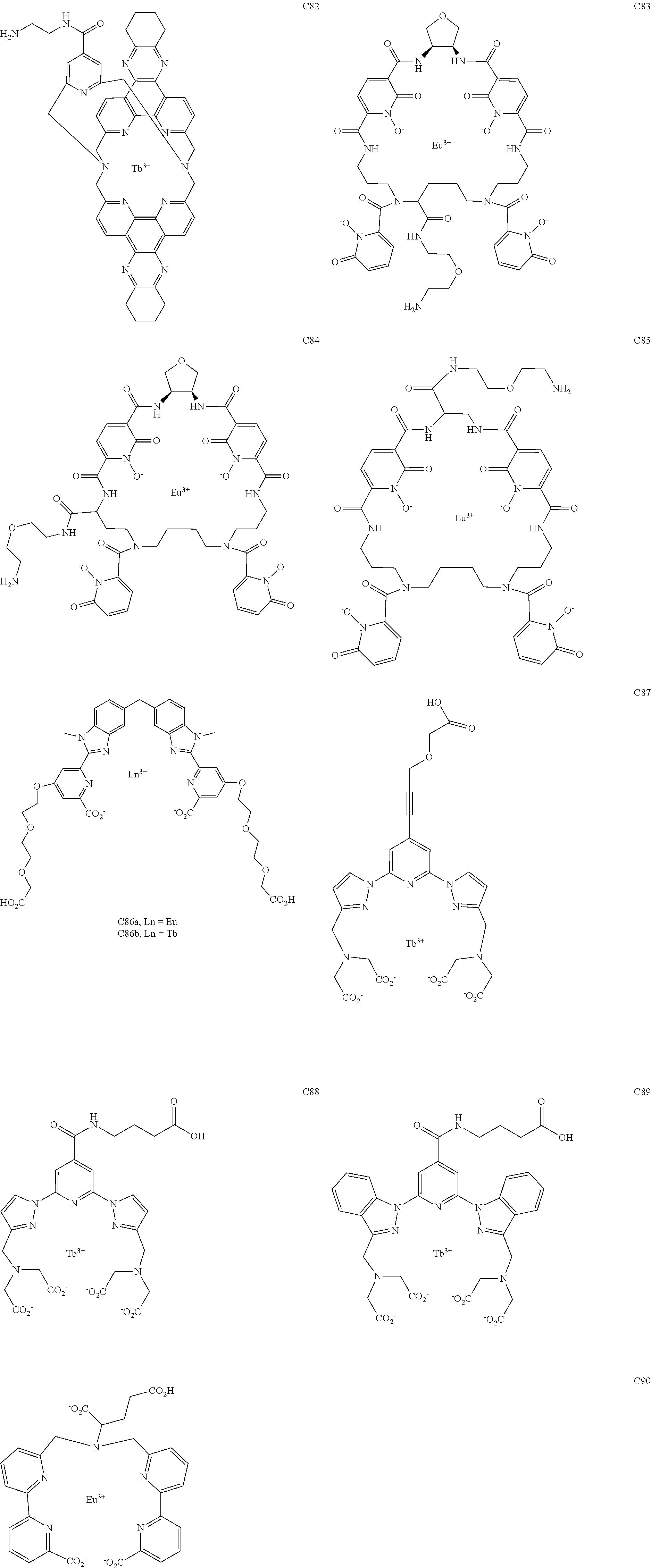

Depending on the pH, the —SO₃H, —CO₂H and —PO(OH)₂ groups are or are not in deprotonated form. These groups thus also denote —SO₃⁻, —CO₂⁻ and —PO(OH)O⁻ groups. Advantageously, the lanthanide complex Ln³⁺ is chosen from one of the complexes C1 to C17, C24 to C32 and C36 to C44. More advantageously, the lanthanide complex Ln³⁺ is chosen from one of the complexes C1 to C17 and C36 to C44. More advantageously still, the lanthanide complex Ln³⁺ is chosen from one of the complexes C1 to C17. More advantageously still, the lanthanide complex Ln³⁺ is chosen from one of the complexes C1 10 to C4 and C11 to C17. More advantageously still, the lanthanide complex Ln³⁺ is chosen from one of the complexes C1 to C4 and C11. Entirely advantageously, the lanthanide complex Ln³⁺ is the complex C2 or the complex C3.

The lanthanide complexes C1 to C90 are described in the publications below. These complexes are either commercially available or can be obtained by the synthesis routes described in said publications.
C1: WO 03/076938
C2: EP-A-0 203 047
C3-C4: WO 2008/025886
C5: Spectrochimica Acta; Part A, Molecular and Biomolecular Spectroscopy, 2001, 57(11), 2197.
C6: Spectrochimica Acta; Part A, Molecular and Biomolecular Spectroscopy, 2001, 57(11), 2197.
C7: Analytical Biochemistry, 2000, 286(1), 17.
C8: WO 01/96877
C9: Spectrochimica Acta; Part A, Molecular and Biomolecular Spectroscopy, 2001, 57(11), 2197.
C10: WO 01/96877
C11: WO 2008/063721
C12-C17: WO 2017/098180.
C18-C20: WO 2013/011236
C21-C35: WO 2014/111661
C36-C44: WO 2018/229408
C45-C47: Organic & Biomolecular Chemistry, 2012, 10, 8509.
C48: WO 2010/084090
C49: WO 2009/010580
C50-C51: Chemical Communications, 2007, 3841
C52: WO 2006/120444
C53: European Journal of Inorganic Chemistry, 2010, 3961.
C54: Bioconjugate Chemistry, 2009, 20(3), 625.
C55: EP-A-0 770 610
C56: WO 2016/106241
C57-C58: EP-A-0 770 610
C59-C60: Analytical Chemistry, 2003, 75, 3193-3201
C61: Bioconjugate Chemistry, 1997, 8(2), 127
C62: WO 93/11433
C63: U.S. Pat. No. 5,696,240
C64a-C81b: WO 2018/22932
C82: WO 2010/070232
C83-C85: WO 2018/045385
C86a-b: Analyst, 2010, 135(1), 42
C87-C89: Chemistry-A European Journal, 2011, 17, 9164
C90: Journal of the American Chemical Society, 2004, 126(15), 4888

The synthesis of the compounds of formula (I) is described in more detail below in schemes 1 to 19. Typically these compounds are obtained by techniques for the conjugation of two organic molecules based on the use of reactive groups, techniques which come within the general knowledge of a person skilled in the art and which are described, for example, in Bioconjugate Techniques, G. T. Hermanson, Academic Press, Second Edition, 2008, pp. 169-211. In order to obtain the GTP-gamma-O compounds, first of all GTP is reacted with a compound of formula G₂-L-G₁, and the intermediate compound thus formed is conjugated with the lanthanide complex. In this formula, G₂-L-G₁:
L is the divalent linking group as defined above;
G₁ is a reactive electrophilic group capable of reacting with the OH functional group of the phosphate at the gamma position of the GTP;
G₂ is a reactive group capable of reacting with a reactive group (G₃) carried by the lanthanide complex Ln³⁺.

The conjugation reaction between the intermediate compound (comprising a reactive group G₂) and the lanthanide complex (comprising a reactive group G₃) results in the formation of a covalent bond comprising one or more atoms of the reactive group.

In one embodiment, the electrophilic group G₁ is:
either an OH group activated by a reactant forming in an intermediate way a reactive entity which can react with the hydroxyl functional group of the phosphate at the gamma position,
or a reactive group of the leaving group type, such as, for example, an aliphatic chloride, an aliphatic bromide, an aliphatic iodide, an aliphatic mesylate, an aliphatic tosylate, an aliphatic triflate, and the like.

In one embodiment, the reactive groups G₂ and G3 are independently of one another chosen from one of the following groups: an acrylamide, an optionally activated amine (for example a cadaverine or an ethylenediamine), an activated ester, an aldehyde, an alkyl halide, an anhydride, an aniline, an azide, an aziridine, a carboxylic acid, a diazoalkane, a haloacetamide, a halotriazine, such as monochlorotriazine or dichlorotriazine, a hydrazine (including hydrazides), an imido ester, an isocyanate, an isothiocyanate, a maleimide, a sulfonyl halide, a thiol, a ketone, an acid halide, a succinimidyl ester, a hydroxysuccinimidyl ester, a hydroxysulfosuccinimidyl ester, an azidonitrophenyl, an azidophenyl, a 3-(2-pyridyldithio)propionamide, a glyoxal, a triazine, an acetylenic group, and in particular a group chosen from the groups of formulae:

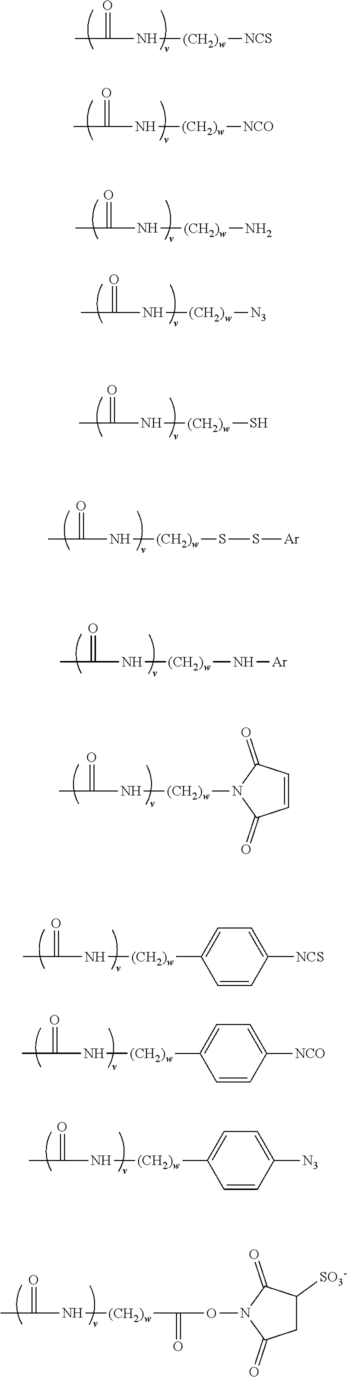

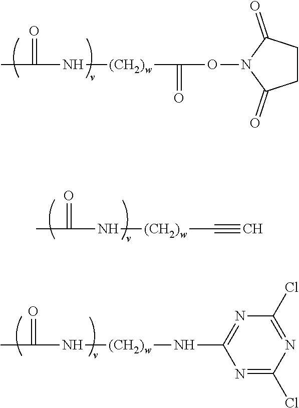

in which w varies from 0 to 8 and v is equal to 0 or 1, and Ar is a saturated or unsaturated 5- or 6-membered heterocycle comprising from 1 to 3 heteroatoms which is optionally substituted by a halogen atom.

G₂ and G₃ can originate from their form protected by a compatible protective group.

Preferably, the reactive groups G₂ and G₃ are independently of one another chosen from an amine (optionally protected in the -NHBoc form), a succinimidyl ester, a hydroxysuccinimidyl ester, a haloacetamide, a hydrazine, a halotriazine, an isothiocyanate, a maleimide group or a carboxylic acid (optionally protected in the form of a —CO₂Me or —CO₂tBu group). In the latter case, the acid will have to be activated in the ester form in order to be able to react with a nucleophilic entity.

In order to obtain GTP-gamma-N compounds, GTP can be reacted directly with a lanthanide complex when the latter has an NH2 group. GTP can also be reacted with a compound of formula ₂HN—(CH₂)ₙ—NH₂ in which n is as defined above and one of the amino groups is optionally protected by a protective group, and then the intermediate compound obtained can be coupled with a lanthanide complex functionalized by a reactive group G₃ as defined above.

The compounds according to the invention are capable of binding to G protein. This property is demonstrated by an immunoassay based on a FRET principle, by incubating a membrane preparation comprising GPCRs and a Gα protein in the presence of a pair of FRET partners consisting of a compound according to the invention and of an anti-Gα protein antibody labeled with an acceptor fluorophore. Incubation is carried out in the presence or absence of a non-hydrolyzable or slowly hydrolyzable GTP analog, such as GTPγS. When the partners of the FRET pair bind to the same Gα protein, a FRET signal appears, thereby demonstrating the binding of the compound of the invention to the Gα protein. The compounds of the invention can thus advantageously be used to identify, by the FRET technique, molecules capable of modulating the activation of a G protein-coupled receptor.

### Synthesis

The syntheses of GTPs coupled in the gamma position to lanthanide complexes are described in schemes 1 to 19.

Synthesis of the Compounds of the GTP-gamma-O (GTPγO) Family

Compound 2, which is a precursor of the compounds of the invention (lanthanide complex GTP), can be synthesized by following the protocols known to a person skilled in the art. Starting from commercially available GTP, the linking group “L1” is introduced at the gamma position of the GTP by nucleophilic substitution between the GTP and the linking group having a leaving group (I, Br, mesyl, tosyl) at the alpha position and a protected amino group on its omega position, thus resulting in compound 1. Analogous coupling examples are available when P=CBz or COCF₃ (cf. WO 2009/105077 or WO 2009/091847). The protective group is removed using the deprotection conditions corresponding to the protective groups (cf. WO 2009/014612). Compound 2 is then covalently coupled via an amide bond to the lanthanide complex using conventional methods known to a person skilled in the art (scheme 1).

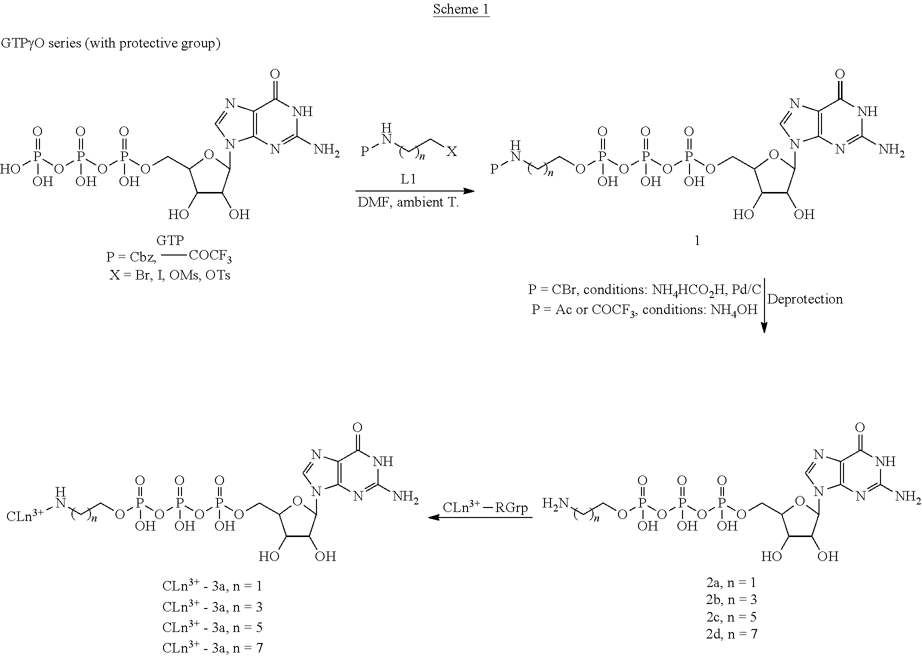

Another alternative for coupling the lanthanide complex at the gamma position of the GTP is the use of “click chemistry”. For this, it is necessary, to begin with, to introduce either an azido group (scheme 2) or an acetylenic group (scheme 3). As above, these groups are introduced via a nucleophilic substitution reaction between the linking group L2 or L3 and the GTP (schemes 2 and 3). Examples of couplings between a nucleotide and a linking group are described, for example, by Hacker et al. (The Journal of Organic Chemistry, 2012, 77(22), 17450).

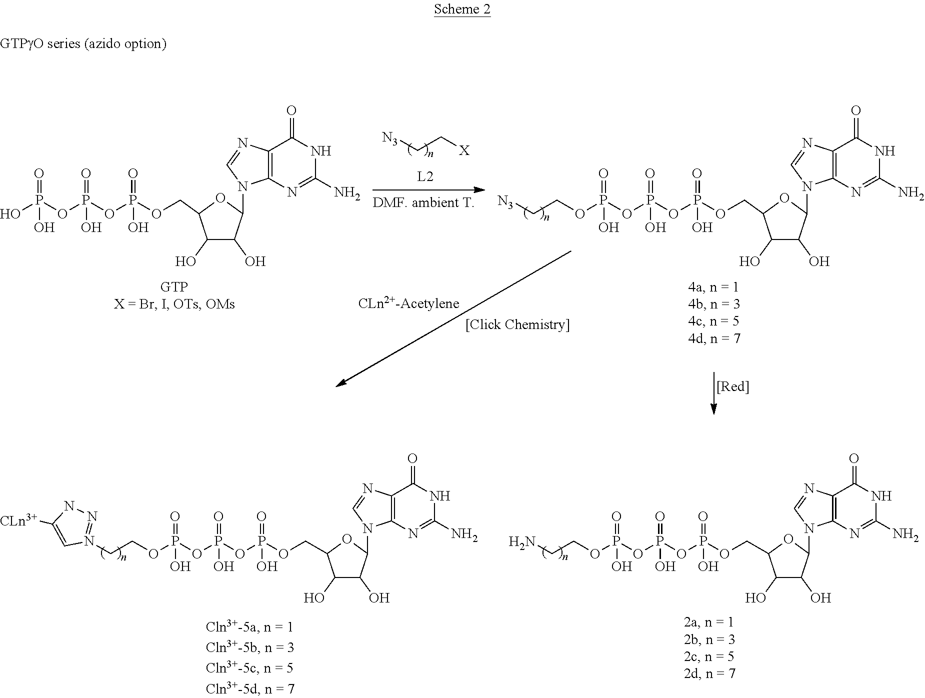

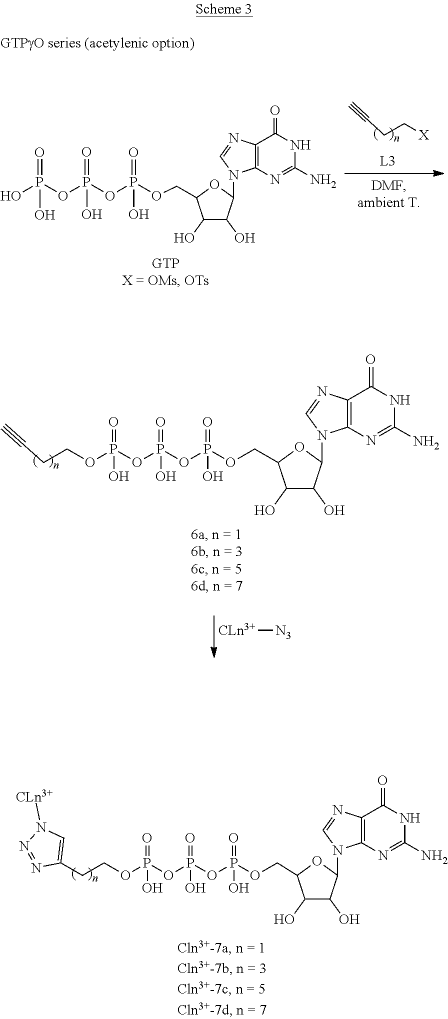

Conversely, the acetylenic group can be introduced in a first stage onto the GTP, resulting in the compounds of the series 6. An example of coupling to other nucleotides is available in the literature (Journal of the American Chemical Society, 2003, 125, 9588). Then the cycloaddition reaction can be carried out with a lanthanide complex functionalized in the azide form to result in the compounds CLn³⁺-7a-d.

### Synthesis of the Compounds of the GTP-gamma-N Family

In the same way as for the GTP-gamma-O family, the GTP-gamma-N compounds can be prepared according to an analogous strategy. The NH₂-functionalized lanthanide complex is condensed directly onto the GTP molecule without intercalation of linker (scheme 4).

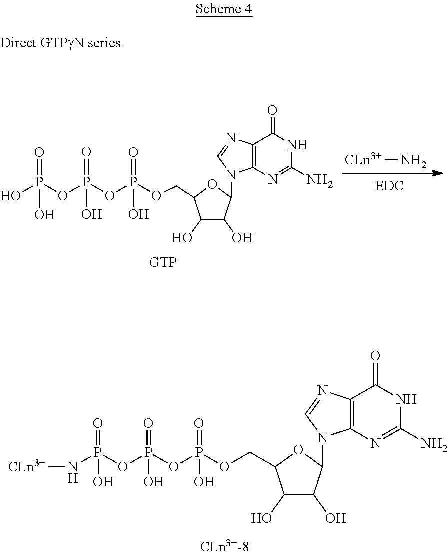

The lanthanide complexes can be coupled using a GTP-gamma-N molecule already comprising a terminal NH₂ linking group previously introduced 9a-e.

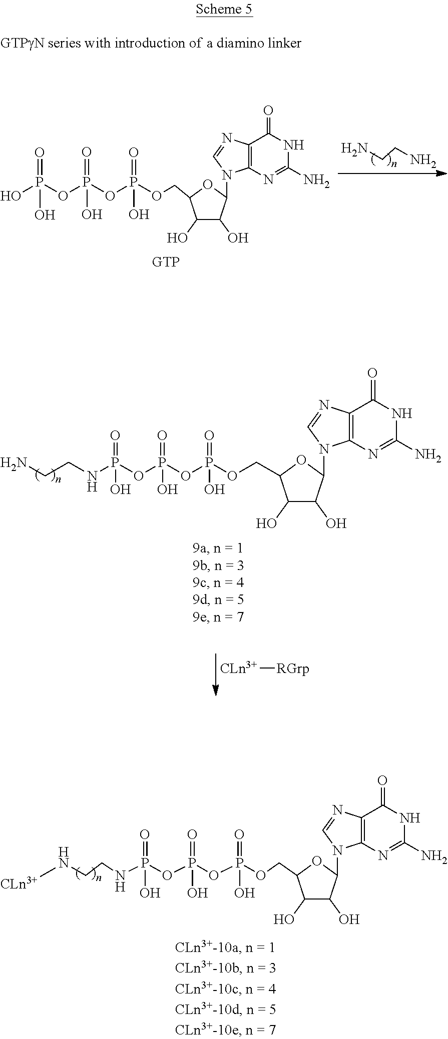

In order to avoid using several equivalents of diamino linking group, one of the functional groups is protected, for example by a CBz or else a trifluoroacetate (COCF₃) group. The protective groups are subsequently removed (under conditions avoiding the chemical hydrolysis of the GTP), thus resulting in the same compounds 9a-e, to which the lanthanide complexes are coupled using conventional techniques known to a person skilled in the art (scheme 6).

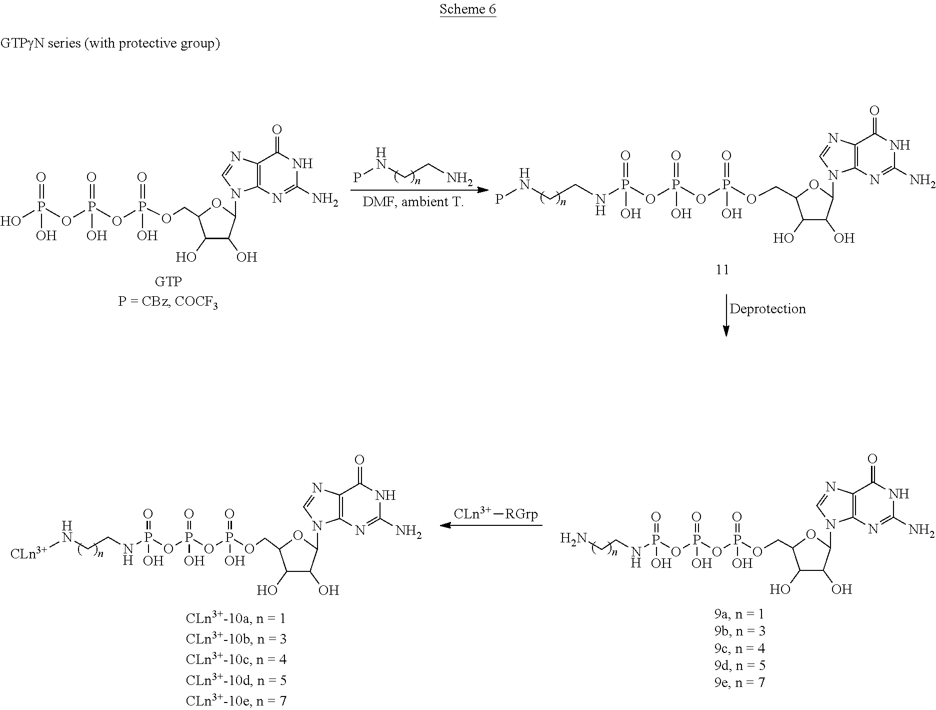

### Synthesis of the Compounds of the GTP-gamma-C Family

The GTP-gamma-C series is synthesized by condensation of GDP (guanosine diphosphate) with phosphonic acids, the terminal position of which can be substituted in different ways. When the terminal position is a protected (trifluoroacetamide 15a-f or CBz 16a-f) amine functional group, these compounds are prepared using the method described in the literature (EP 0 959 077 (TFA); WO 2012/150866 and The Journal of Organic Chemistry, 1984, 49, 1158 (CBz)). N-Phthalimidoalkyl bromides (12a-f) are condensed with triethyl phosphite in a first stage, subsequently followed by treatment with hydrazine to deprotect the amine functional group. The ethyl phosphonates are hydrolyzed in the presence of hydrobromic acid to result in compounds 14a-f. The primary amine functional group is protected either by a CBz or by a trifluoroacetamide in order to prevent a self-condensation reaction during the coupling reaction with GDP. This reaction sequence makes it possible to obtain the intermediate compounds 15a-f and 16a-f.

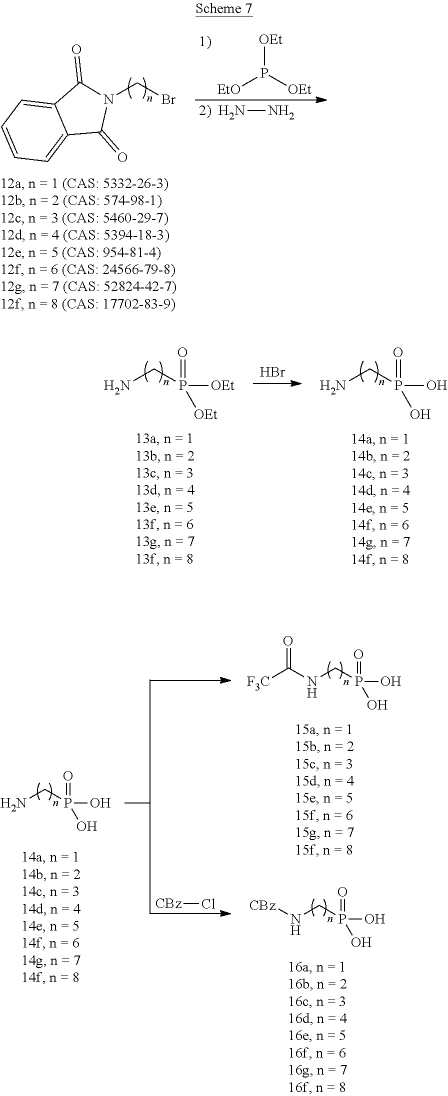

Instead of introducing a masked primary amine functional group, it is also possible to have available GTP-gamma-C, the terminal part of the linker of which is an acetylenic unit. This functional group makes it possible to carry out a “Click Chemistry” reaction between the acetylenic GTP-gamma-C and a lanthanide complex carrying an azide functional group available for a coupling reaction. Scheme 8 briefly describes the various intermediates prepared according to the protocols available in the literature (Bioorganic Medicinal Chemistry, 2018, 26, 191, and Angewandte Chemie International Edition, 2011, 50, 10699). The commercial acetylenic alkyl bromide derivatives are condensed with trimethylsilyl phosphite, then hydrolyzed to result in the 18a-f series.

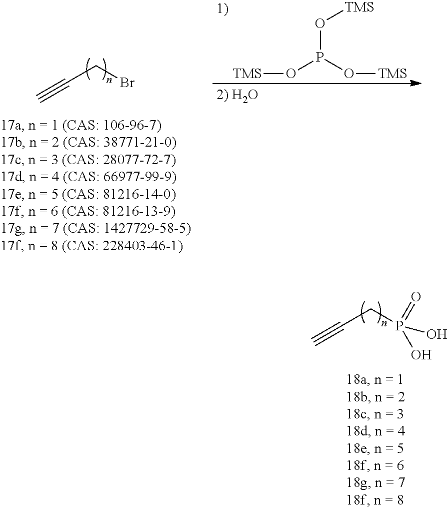

Another option is possible in order to introduce, onto the linker of the GTP-gamma-C compounds, a functional group making possible bioconjugation. In this approach, the azido group is introduced onto the phosphonic acid using the sequence described in scheme 9. The protocols are described in the literature, for example in Chemistry, A European Journal, 2010, 16, 12718, Langmuir, 2010, 26, 10725, or indeed also in The Journal of Organic Chemistry, 2012, 77, 10450. The dibrominated derivatives react with triethyl phosphite to give compounds 20a-f. The azido functional group is subsequently introduced by a simple nucleophilic substitution with sodium azide. The diesters are subsequently hydrolyzed in the presence of TMS-Br, which results in derivatives 22a-f.

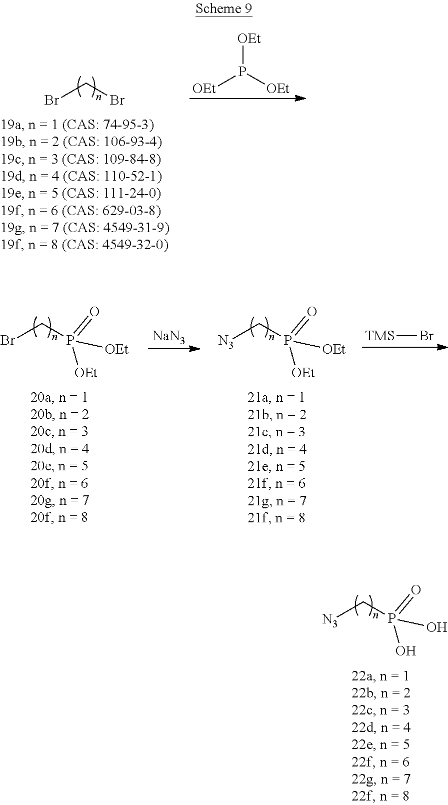

The coupling of compounds 15a-f, 16a-f, 18a-f or 22a-f with the lanthanide complexes is carried out using conventional techniques known to a person skilled in the art (schemes 10 to 13).

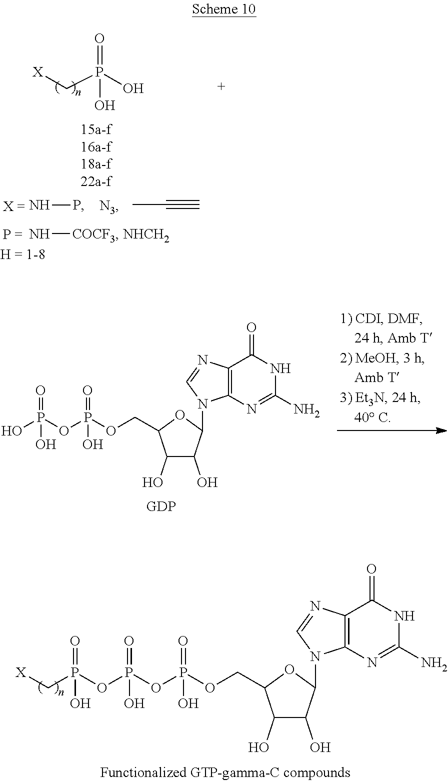

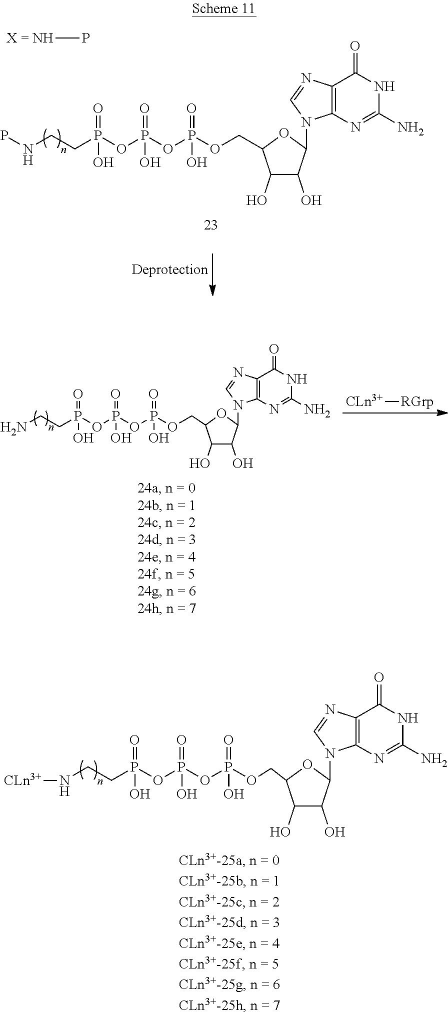

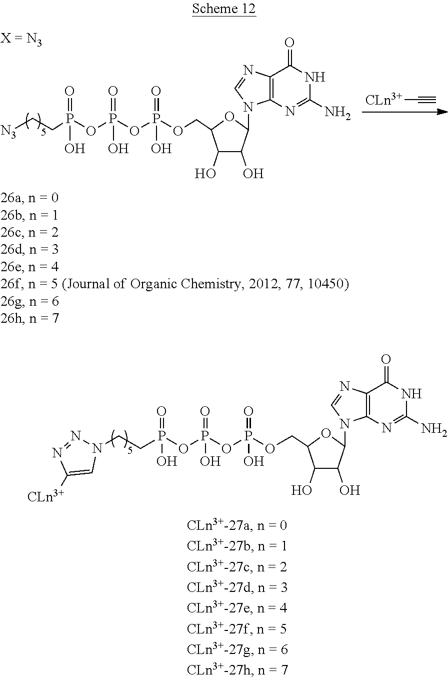

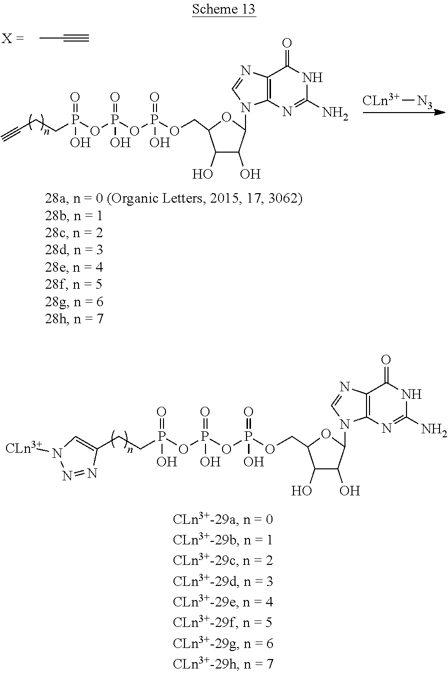

In the same way as for the GTPs, the analog GPPNHP (commercial, CAS: 64564-03-0) can be coupled with lanthanide complexes at the gamma position using the same synthesis strategies. For the derivatives substituted at gamma O, the synthesis is described, for example, in scheme 14. For the derivatives substituted at gamma N, the syntheses are described in schemes 15 and 16. These couplings have also been exemplified in the application WO 2009/068751, which uses ATP analogs.

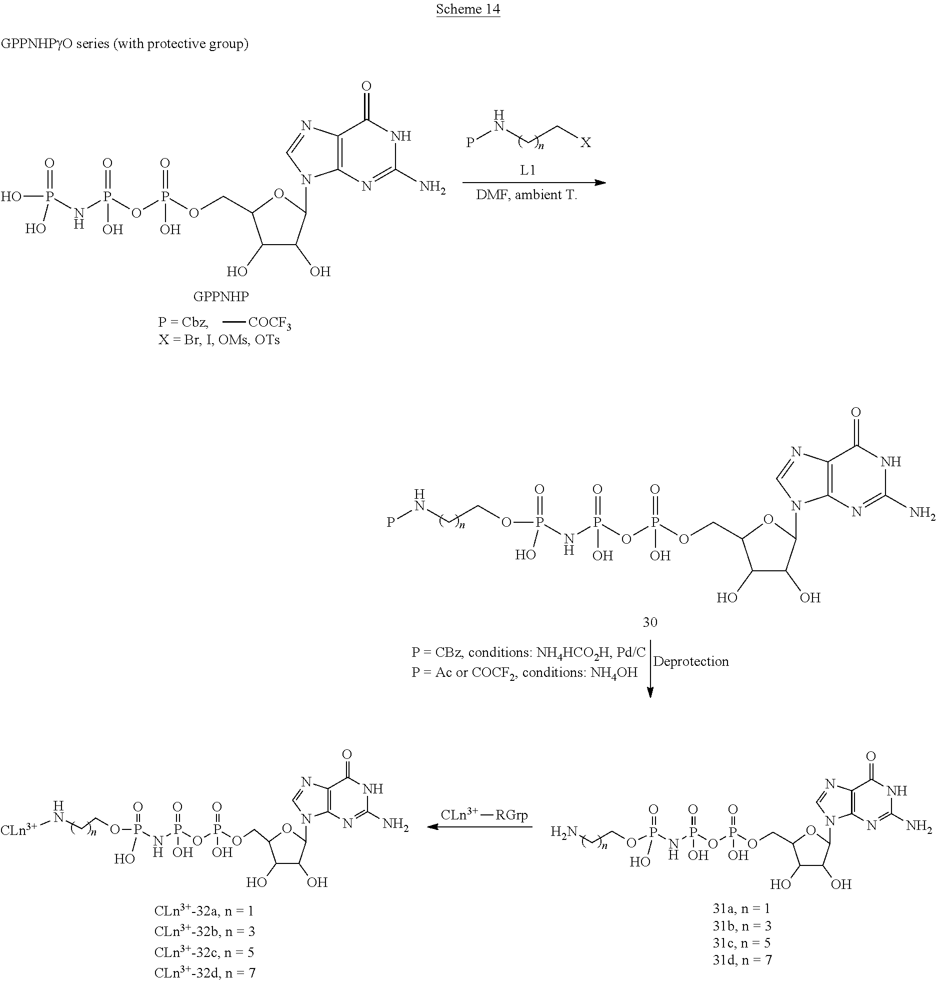

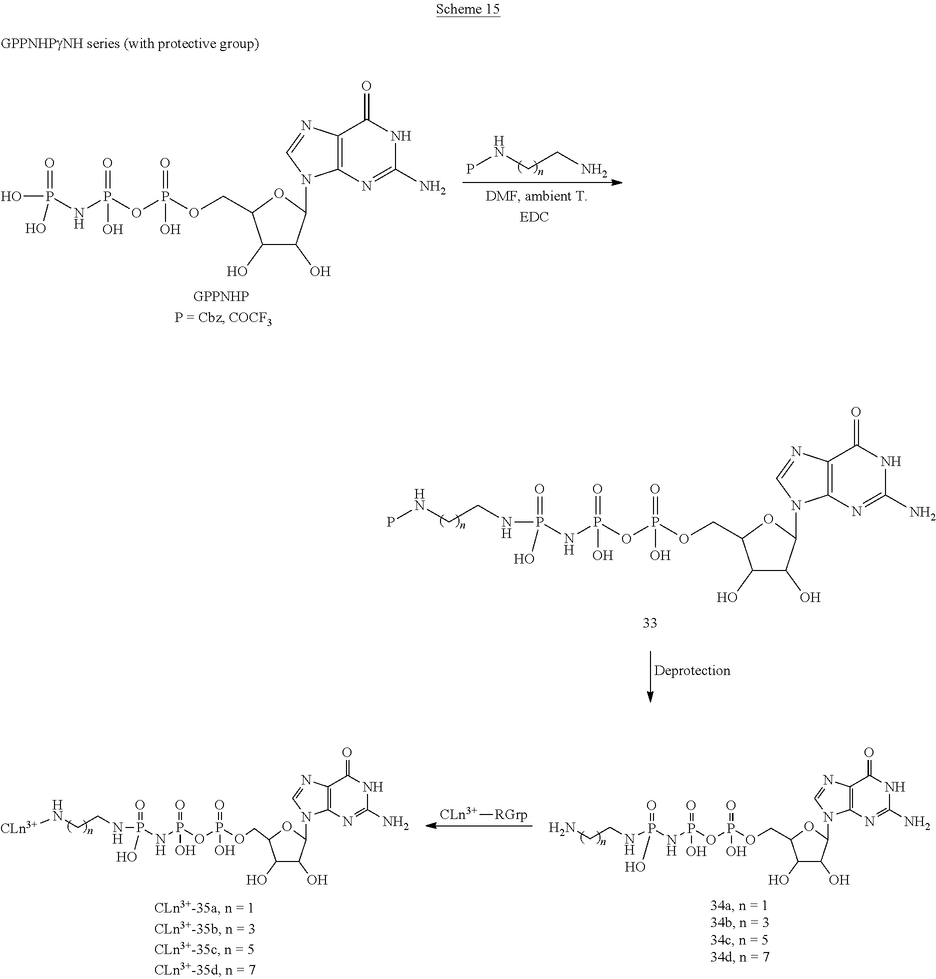

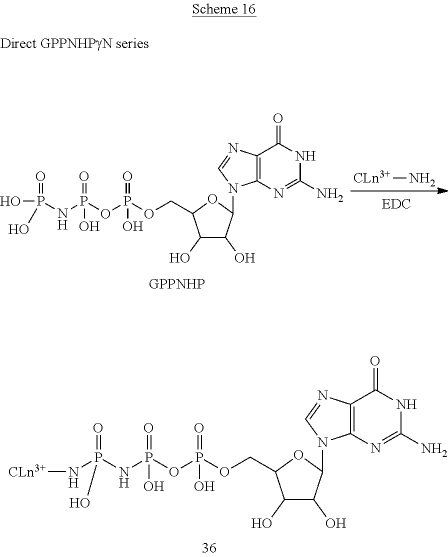

In the same way as for the GTPs, the GPPCH₂P analog (commercial, CAS: 13912-93-1 or 10470-57-2 in the form of the Na salt) can be coupled with lanthanide complexes in the gamma position using the same synthesis strategies. For the derivatives substituted at gamma O, the synthesis is described, for example, in scheme 17. For the derivatives substituted at gamma N, the syntheses are described in schemes 18 and 19. These couplings have also been exemplified in the application WO 2009/068751, which uses ATP analogs.

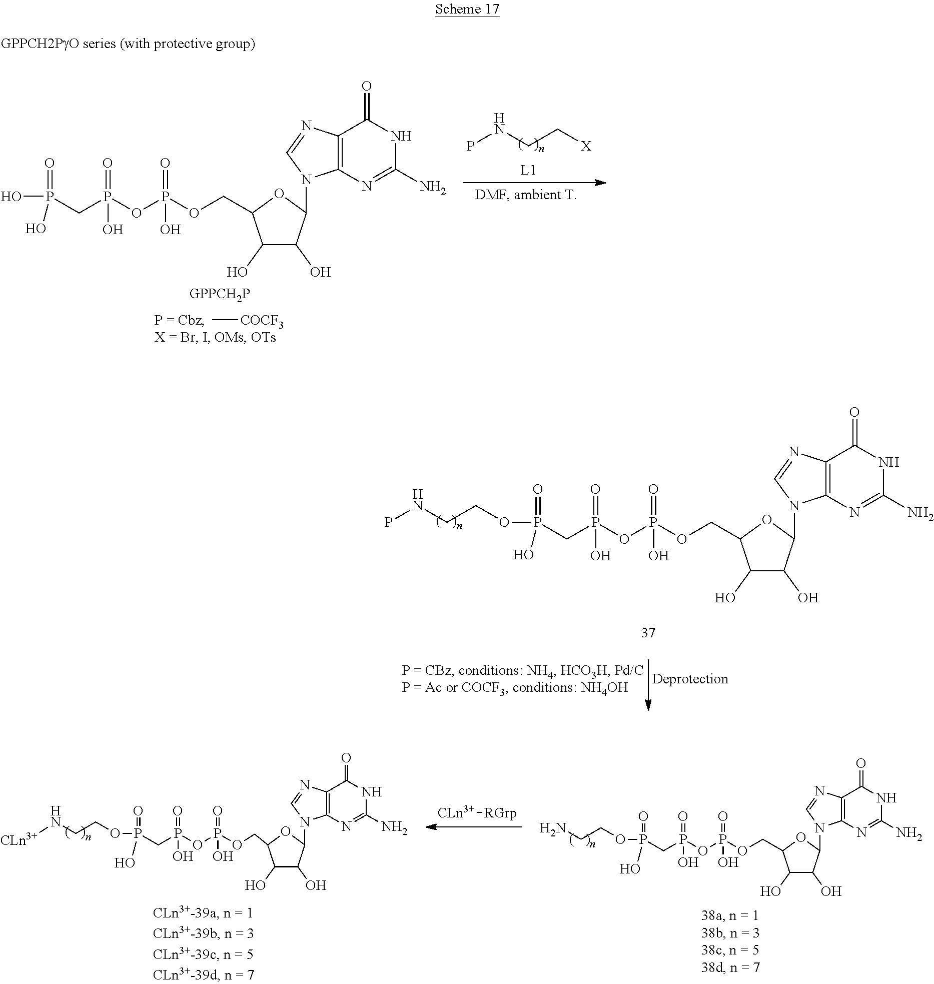

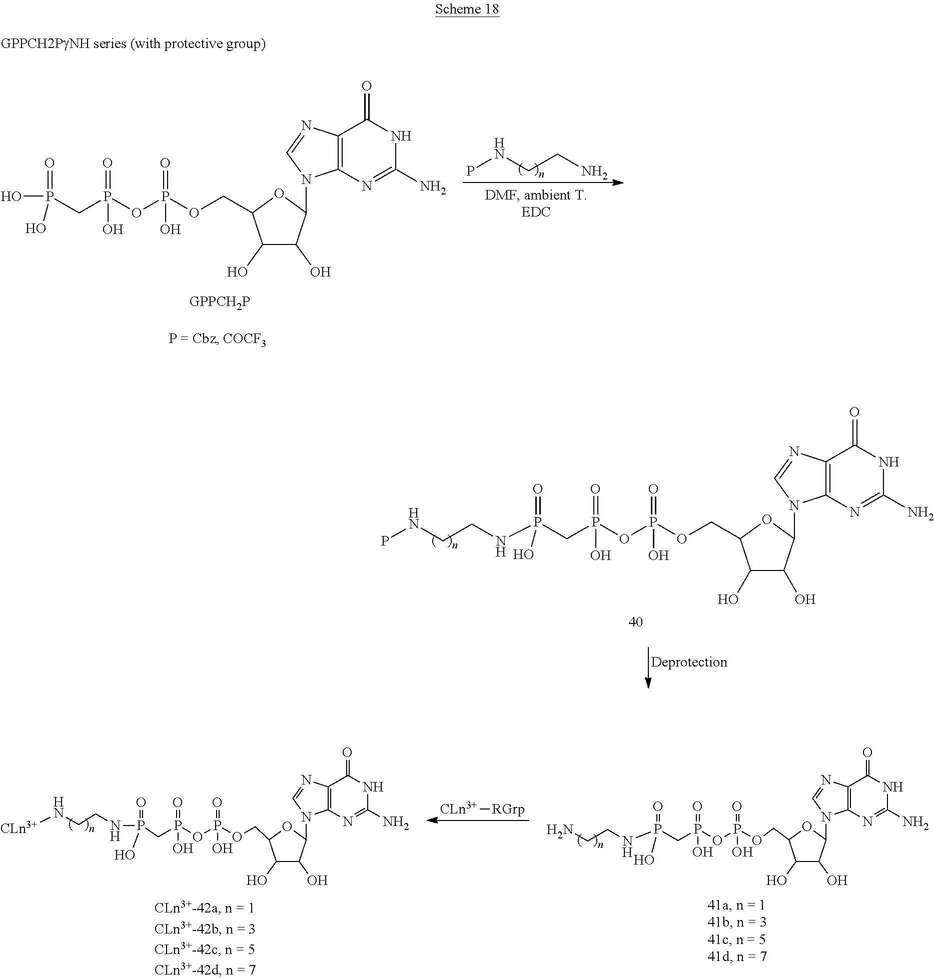

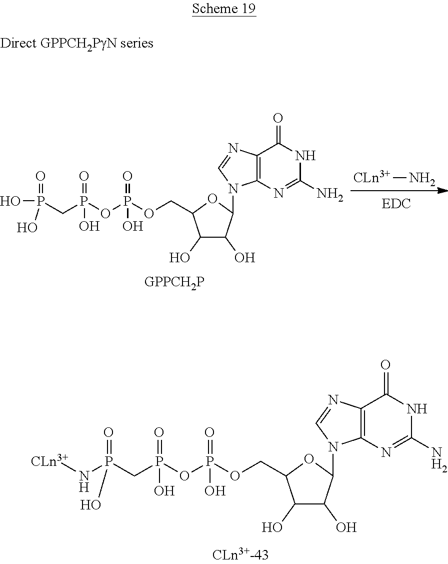

The invention is illustrated by the examples below, given by way of illustration.

### Abbreviations Used

BRET: Bioluminescence Resonance Energy Transfer
BSA: Bovine Serum Albumin
DMSO: dimethyl sulfoxide
EDC: N-(3-dimethylaminopropyl)-N′-ethylcarbodiimide
eq: equivalent
ESI+: positive electrospray ionization mode
FRET: Förster Resonance Energy Transfer
GDP: guanosine diphosphate
GPCR: G protein-coupled receptors
GTP: guanosine triphosphate
GTPγS: guanosine 5′-[γ-thio]triphosphate
h: hour
HEPES: 4-(2-hydroxyethyl)-1-piperazineethanesulfonic acid
HPLC: high performance liquid chromatography
LC-MS: high performance liquid chromatography coupled with mass spectrometry
LRMS: low resolution mass spectrometry
MES: 2-(N-morpholino)ethanesulfonic acid
MOPS: 3-morpholino-1-propanesulfonic acid
NCS: isothiocyanate
NHS: N-hydroxysuccinimide
Py: pyridine
GPCR: G protein-coupled receptors
AT: ambient temperature
TEA: triethylamine
TRIS: trishydroxymethylaminomethane
UPLC: ultra high performance liquid chromatography
UPLC-MS: ultra high performance liquid chromatography coupled with mass spectrometry
UV: Ultraviolet

### Chromatography

Analytical and preparative high performance liquid chromatography (HPLC) procedures were carried out on two appliances:

Analytical HPLC: ThermoScientific, P4000 quaternary pump, UV 1000 detector having a deuterium lamp (190-350 nm), Waters XBridge C₁₈, 3.5 μm, 4.6×100 mm, analytical column.

Preparative HPLC: Shimadzu, 2 LC-8A pumps, Varian ProStar diode array UV detector, Waters XBridge prep. C₁₈, 5 μm: 19×100 mm or 50×150 mm, preparative column.

Analytical ultra-high performance liquid chromatography (UPLC) procedures were carried out on a Waters Acquity HClass appliance with, as detector, either a PDA-type diode array UV detector or an SQD2-type single quadrupole mass detector. The probe used is an electrospray in positive mode: capillary voltage at 3.2 kV—cone voltage at 30 V.

Gradient A Waters Xbridge C₁₈, 5 μm, 10×100 mm, column, A/water 25 mM triethylammonium acetate pH 7−B/acetonitrile−t=0 to 5 min 1% B−t=10 min 10% B−20 ml·min⁻¹ at 260 nm.

### Gradient B

Waters Xbridge C₁₈, 5 μm, 4.6×100 mm, column, A/water 25 mM triethylammonium acetate pH 7−B/acetonitrile−t=0 to 2 min 2% B−t=19 min 40% B−1 ml·min⁻¹.

### Gradient C

Waters Xbridge C₁₈, 3.5 μm, 4.6×100 mm, column-A/water 5 mM ammonium acetate pH 5−B/acetonitrile t=0 min 2% B−t=1 min 2% B−t=15 min 40% B−1 ml·min⁻¹.

### Gradient D

Waters Xbridge C₁₈, 5 μm, 19×100 mm, column-A/water 5 mM ammonium acetate pH 6.6−B/acetonitrile t=0 min 2% B−t=2.5 min 2% B−t=27 min 40% B−12 ml·min⁻¹ at 280 and 320 nm.

### Gradient E

Waters Xbridge C₁₈, 5 μm, 19×100 mm, column-A/water 25 mM triethylammonium acetate pH 7−B/acetonitrile t=0 min 2% B−t=2 min 2% B−t=20 min 50% B−20 ml·min⁻¹ at 280 and 320 nm.

### Gradient F

Waters Acquity C₁₈, 300 Å, 1.7 μm, 2.1×50 mm, column-A/water 5 mM ammonium acetate pH 5−B/acetonitrile t=0 min 2% B−t=0.2 min 2% B−t=5 min 40% B−0.6 ml·min⁻¹.

### Gradient G

Waters Xbridge C₁₈, 300 Å, 5 μm, 10×100 mm, column-A/water 25 mM triethylammonium acetate−B/acetonitrile t=0 min 2% B−t=19 min 20% B−5 ml·min⁻¹.

### Gradient H

Waters Xbridge C₁₈, 5 μm, 4.6×100 mm, column, A/water 25 mM triethylammonium acetate pH 7−B/acetonitrile−t=0 to 2 min 2% B−t=19 min 50% B−1 ml·min⁻¹.

### Gradient I

Waters Xbridge C₁₈, 5 μm, 10×100 mm, column-A/water 25 mM triethylammonium acetate pH 7−B/acetonitrile t=0 min 2% B−t=2 min 2% B−t=27 min 50% B−3.5 ml·min⁻¹ at 280 nm.

### Gradient J

Waters Xbridge C₁₈, 5 μm, 19×100 mm, column-A/water 25 mM triethylammonium acetate−B/acetonitrile t=0 min 5% B−t=17 min 45% B−20 ml·min⁻¹.

### Gradient K

Waters Xbridge C₁₈, 300 Å, 5 μm, 10×100 mm, column-water 25 mM triethylammonium acetate−B/acetonitrile t=0 min 2% B−t=19 min 40% B−5 ml·min⁻¹.

### Preparation of the Compounds

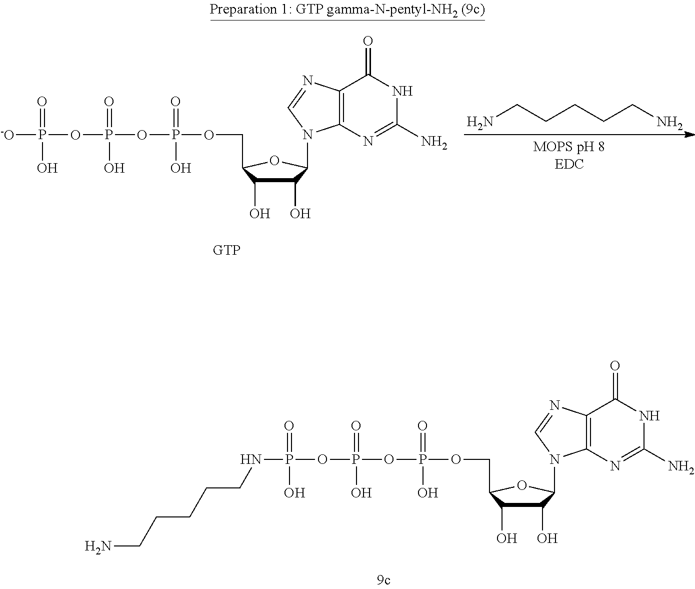

EDC (40 mg; 210 μmol, 11 eq) and a solution of cadaverine (58 mg, 573 μmol, 30 eq) in MOPS at pH 8 were added to a round-bottomed flask containing GTP (10 mg; 19.1 μmol, 1 eq) in solution in water (500 μl). The reaction mixture was stirred at AT overnight before being purified by preparative HPLC (Gradient A). A white solid was obtained corresponding to the compound 9c (5 mg). (ESI+): calculated C₁₅H₂₈N₇O₁₃P₃ [M+H]⁺, m/z=608.10, found 608.55.

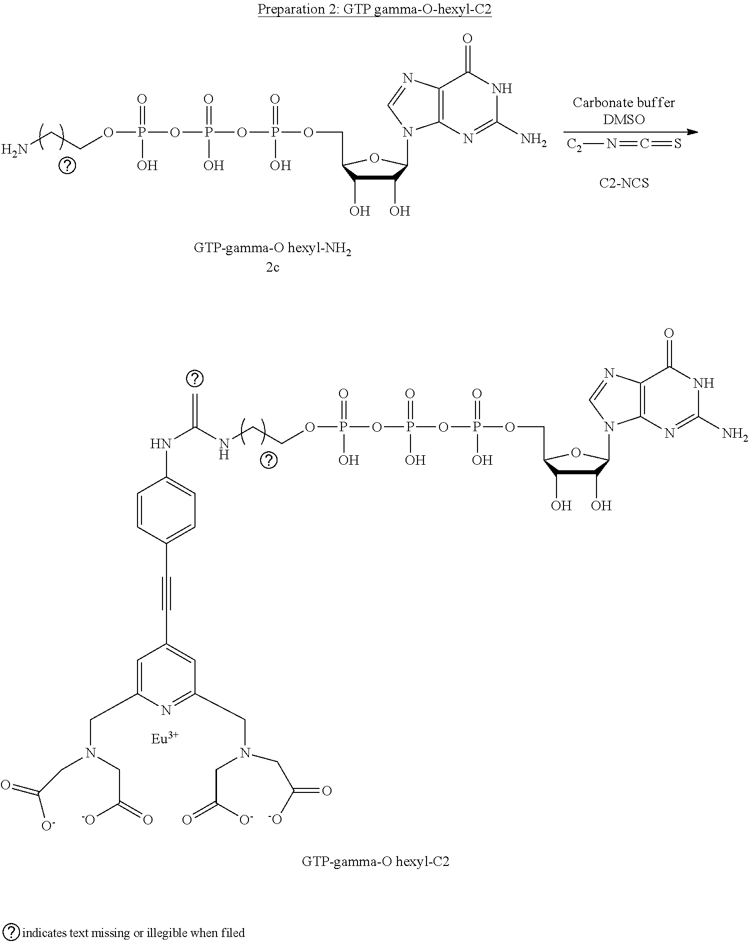

Complex C2 functionalized in its NCS (isothiocyanate) form (473 μg, 700 nmol) in solution in DMSO (200 μl) was added all at once to a solution of compound 2c (0.454 mg, 700 nmol) in 100 mM carbonate buffer pH 9 (500 μl). The mixture was stirred at AT overnight. The progress of the reaction was monitored by HPLC (Gradient B) and UPLC-MS (Gradient C); after this period, the reaction was complete. The reaction mixture was directly purified by semi-preparative HPLC (Gradient D) to result in the compound GTP-gamma-O-hexyl-C2 (125 μg, 96 nmol, 14%) in the form of a white powder. LRMS (ESI+): calculated for C₄₀H₄₇EuN₁₀O₂₂P₃S⁻ [M+3H]²⁺, m/z=650.06, found 650.32.

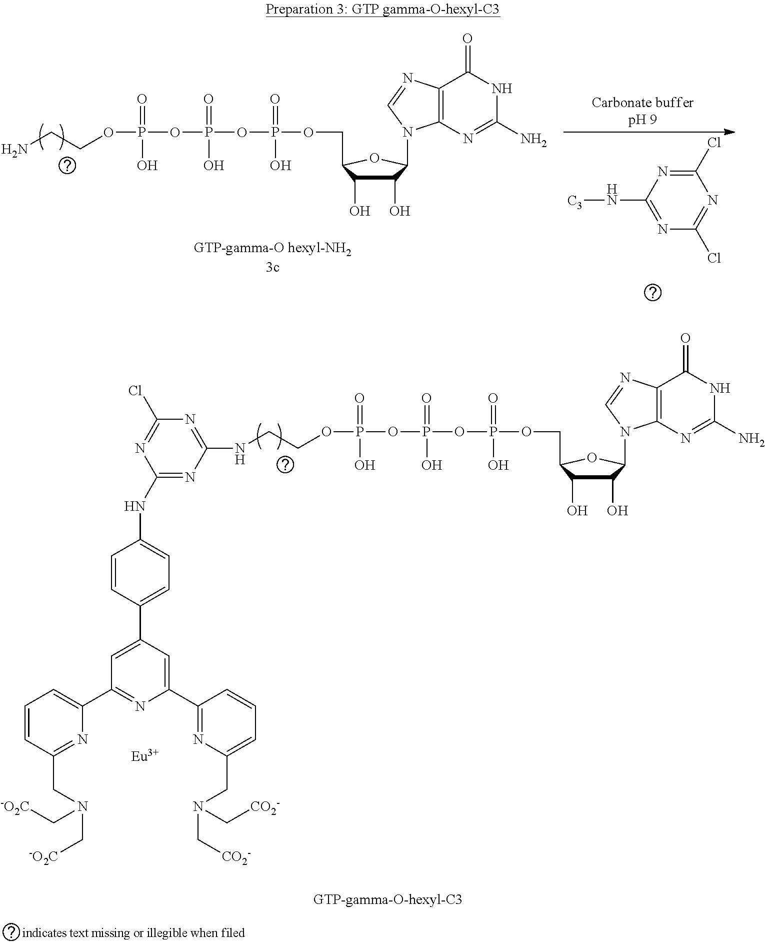

Complex C3 functionalized in its dichlorotriazine form (0.647 mg, 708 nmol) in solution in 100 mM carbonate buffer pH 9 (200 μl) was added all at once to a solution of compound 2c (0.442 mg, 710 nmol) in 100 mM carbonate buffer pH 9 (200 μl). The mixture was stirred at 4° C. overnight. The progress of the reaction was monitored by UPLC-MS (Gradient C); after this period, the reaction was complete. The reaction mixture was directly purified by preparative HPLC (Gradient E) to result in the compound GTP-gamma-O-hexyl-C3 (0.11 mg, 70 nmol, 10%) in the form of a white powder. LRMS (ESI+): calculated for C₅₀H₅₃ClEuN₁₅O₂₂P₃ [M+3H]²⁺, m/z=749.59, found 750.29.

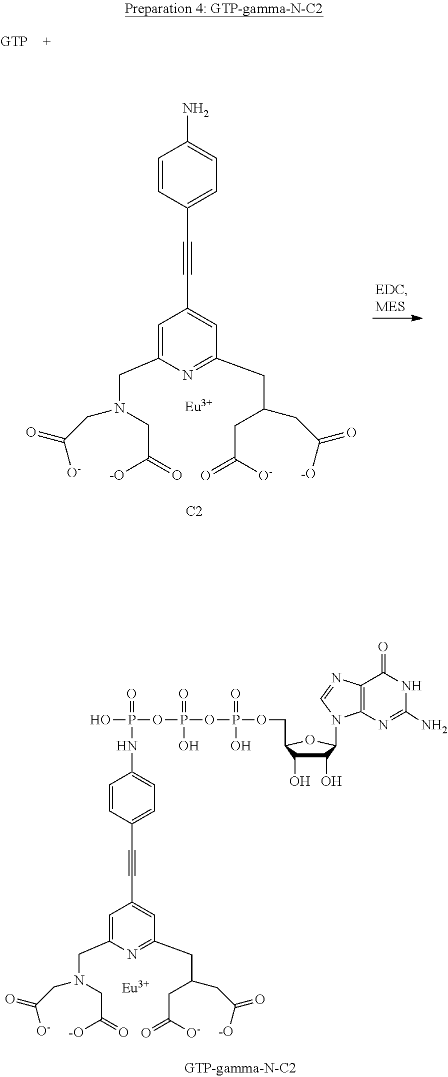

Complex C2 (0.366 mg, 2 μmol) was dissolved in 500 mM MES buffer pH 5.5 (150 μl) to give a yellow solution. GTP (1.32 mg, 2.4 μmol) and then EDC (1.92 mg, 10 μmol) all at once were added to the reaction mixture. The mixture was stirred at AT for 40 h. The progress of the reaction was monitored by UPLC-MS (Gradient F); after this period, the reaction was partial and no longer progressed. The reaction mixture was directly purified by preparative HPLC (Gradient G) to result in GTP-gamma-N-C2 (60 nmol, 3%) in the form of a colorless oil. LRMS (ESI+): calculated for C₃₃H₃₄N₉O₂₁P₃Eu⁻ [M]⁻, m/z=1137.6, found 1138.2.

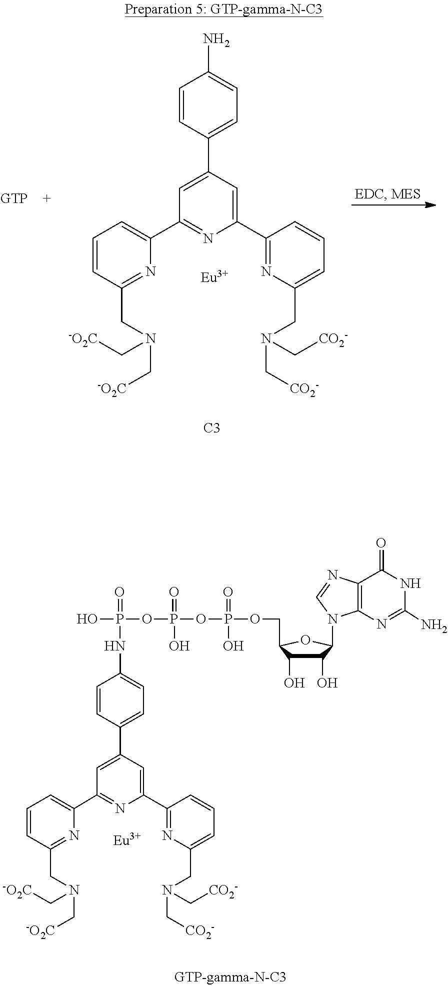

Complex C3 (0.366 mg, 480 nmol) was dissolved in 500 mM MES buffer pH 5.5 (150 μl) to give a yellow solution. GTP (0.317 mg, 576 nmol) and then EDC (0.460 mg, 2.4 μmol) all at once were added to the reaction mixture. The mixture was stirred at AT overnight. The progress of the reaction was monitored by UPLC-MS (Gradient F); after this period, the reaction was partial and no longer progressed. The reaction mixture was directly purified by preparative HPLC (Gradient G) to result in GTP-gamma-N-C3 (45.6 nmol, 9.5%) in the form of a colorless oil. LRMS (ESI+): calculated for C₄₁H₄₀N₁₁O₂₁P₃Eu⁻ [M+2H]⁺, m/z=1269.7, found 1269.

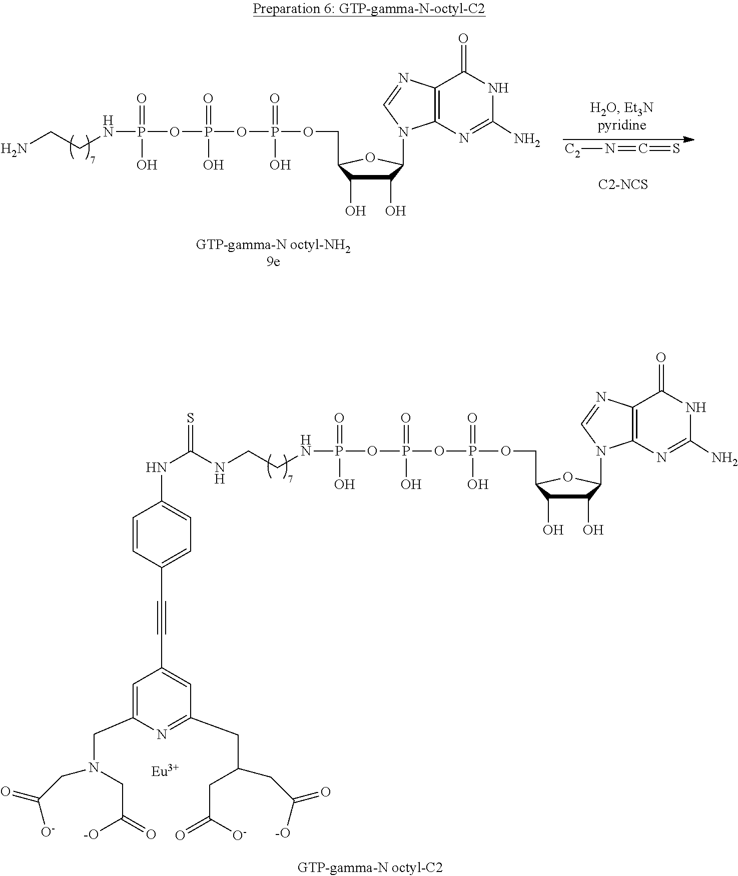

Compound 9e (50.0 μl, 500 nmol) was dissolved in water (100 μl). Pyridine (300 uμl) and triethylamine (6 μl) were added to the reaction mixture. The reaction mixture was added to a tube containing complex C2 functionalized in its NCS (isothiocyanate) form (0.438 mg, 650 nmol) all at once. The mixture was stirred at 24° C. for 12 h. The progress of the reaction was monitored by UPLC-MS (Gradient F); after this period, the reaction was partial. The reaction mixture was directly purified by preparative HPLC (Gradient G) to result in GTP-gamma-N-octyl-C2 (16.9 nmol, 3.4%) in the form of a colorless oil. LRMS (ESI+): calculated for C42H52N11O21P3SEu⁻ [M+3H]2+-, m/z=663.5, found 663.9.

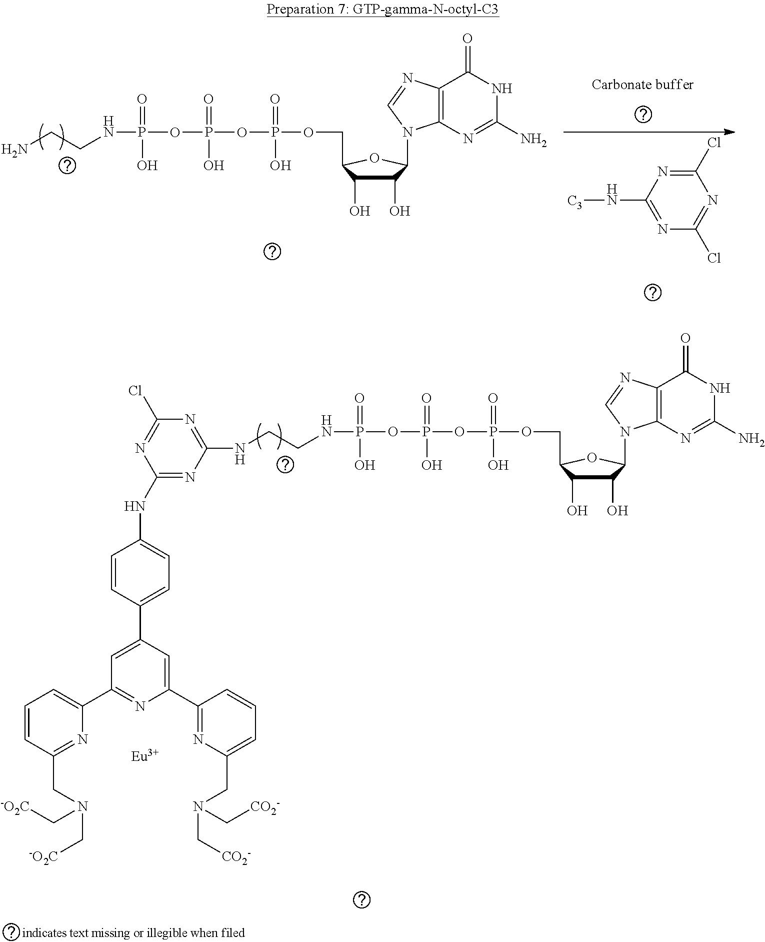

Complex C3 functionalized in its dichlorotriazine form (400 μg, 438 nmol) in solution in water (200 μl) was added all at once to a solution of compound 9e (0.545 mg, 840 nmol) in 100 mM carbonate buffer pH 9 (500 μl). The mixture was stirred at 20° C. overnight. The progress of the reaction was monitored by HPLC (Gradient H) and UPLC-MS (Gradient C). After this period, the reaction was complete. The reaction mixture was directly purified by semi-preparative HPLC (Gradient I) to result in the compound GTP-gamma-N-octyl-C3 (7.6 μg, 5 nmol, 1%) in the form of a white powder. LRMS (ESI+): calculated for C₅₂H₅₈ClEuN₁₆O₂₁P₃⁻ [M+3H]²⁺, m/z=763.11, found 763.29.

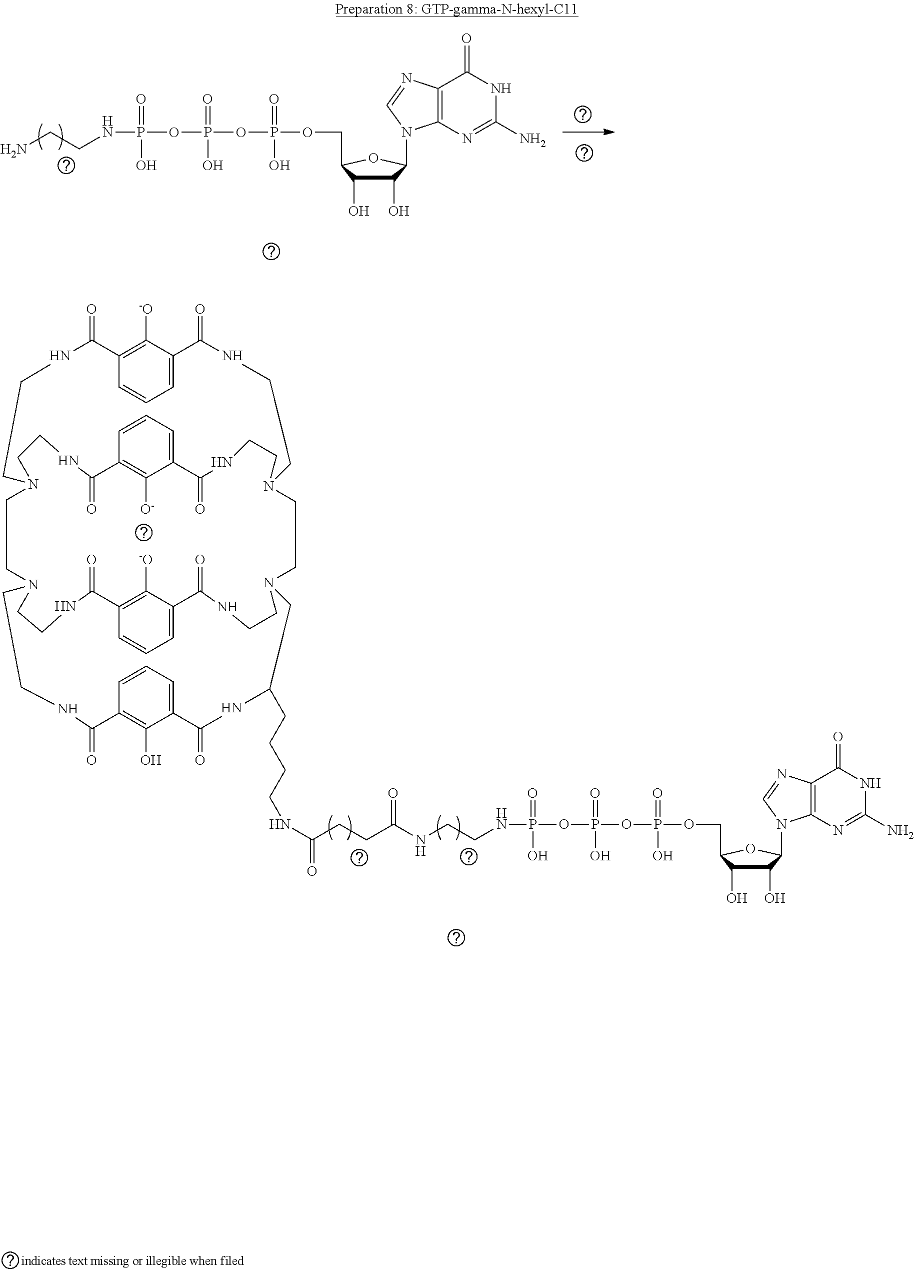

Complex C11 functionalized in the NHS ester form (1.47 mg, 1 μmol) in solution in anhydrous DMSO (122 μl) was added all at once to a solution of compound 9d (0.62 mg, 1 μmol) in 50 mM HEPES buffer pH 8 (900 μl). The mixture was stirred at 24° C. for 1 h. The progress of the reaction was monitored by UPLC-MS (Gradient F); after this period, the reaction was complete. The reaction mixture was directly purified by preparative HPLC (Gradient J) to result in the compound GTP-gamma-N-hexyl-C11 (0.52 mg, 258 nmol, 26%) in the form of a white powder. LRMS (ESI+): calculated for C₈₀H₁₀₉N₂₀O₂₇P₃ Tb³⁺ [M−H]²⁺, m/z=1018.9, found 1019.6, [M+2Na⁺−3H]2+-, m/z=1040.8, found 1041.6.

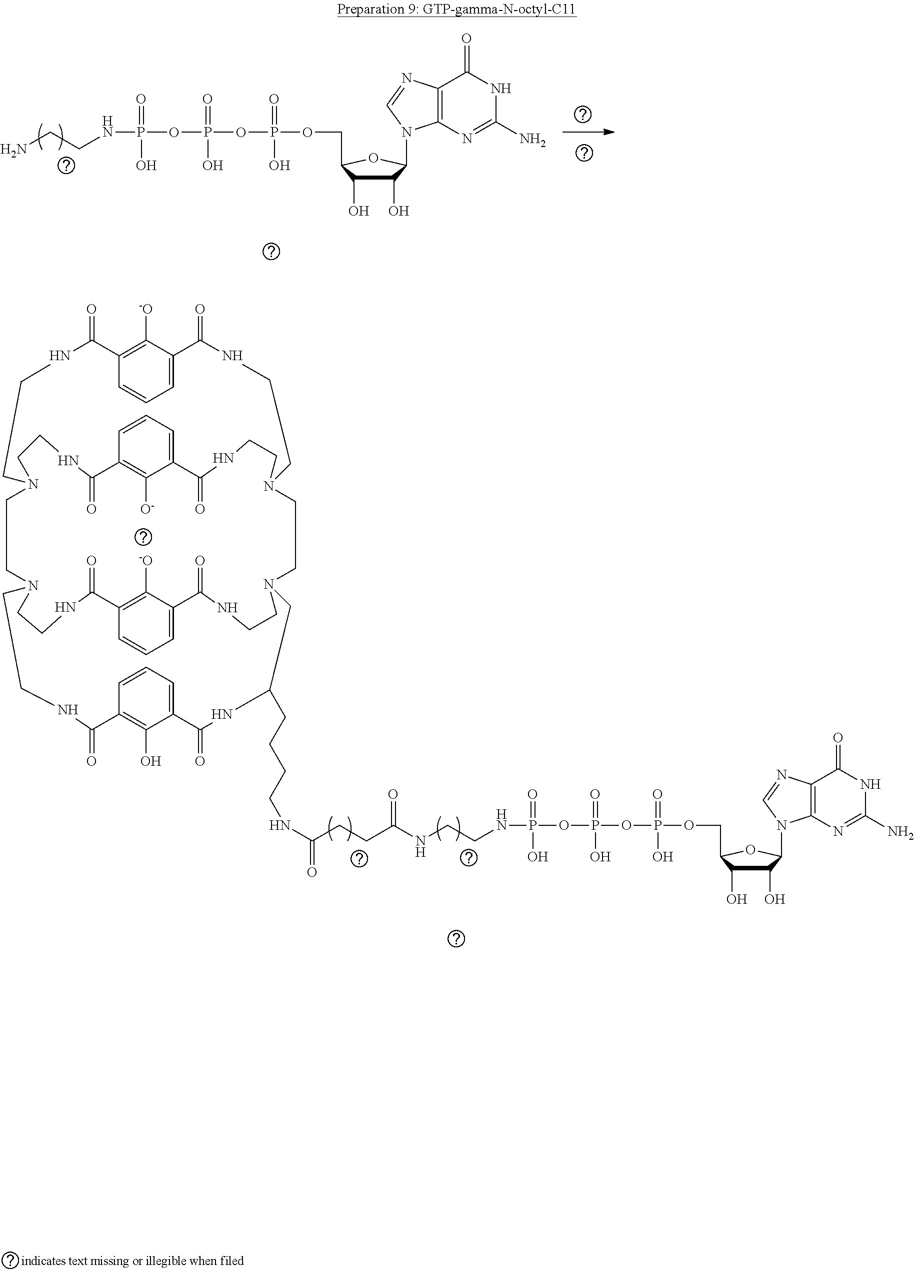

Complex C11 functionalized in the NHS ester form (0.76 mg, 500 nmol) in solution in anhydrous DMSO (102 μl) was added all at once to a solution of compound 9e (0.32 mg, 500 nmol) in 50 mM HEPES buffer pH 8 (300 μl). The mixture was stirred at 24° C. for 1 h. The progress of the reaction was monitored by UPLC-MS (Gradient F); after this period, the reaction was complete. The reaction mixture was directly purified by preparative HPLC (Gradient K) to result in the compound GTP-gamma-N-octyl-C11 (0.22 mg, 105 nmol, 21%) in the form of a white powder. LRMS (ESI+): calculated for C₈₂H₁₁₇N₂₀O₂₇P₃Tb³⁺ [M−2H]⁺, m/z=2064.8, found 2064.1.

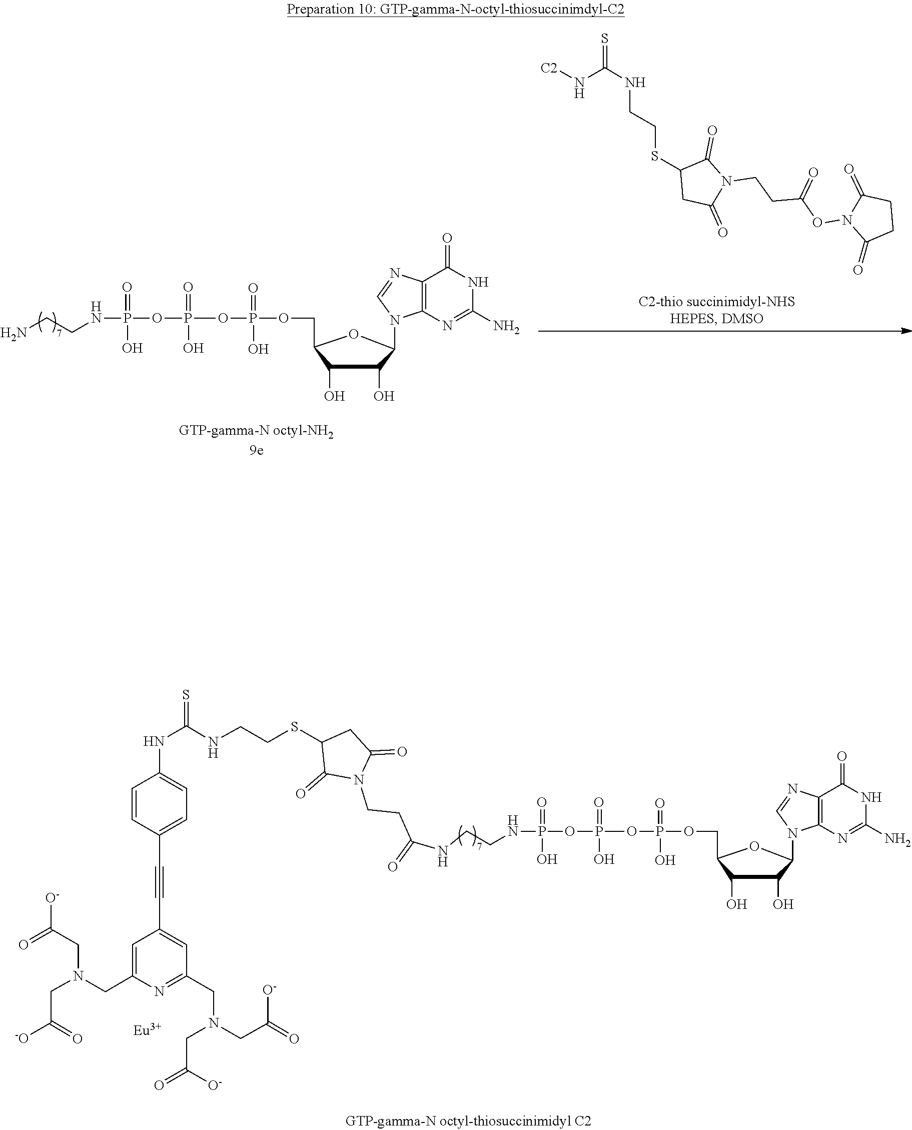

Compound 9e (0.649 mg, 1 μmol) in solution in water (100 μl) was diluted in 50 mM HEPES buffer pH 8 (757 μl) to give a colorless solution. Complex C2 functionalized in its NHS form (N-hydroxysuccinimide ester) (750 μl, 1 μmol) in solution in anhydrous DMSO (143 μl) was added to the reaction mixture in one go. The reaction was stirred at 25° C. for 1 h. The progress of the reaction was monitored by UPLC-MS (Gradient B). After this period, the reaction was not complete. Despite this, the reaction mixture was purified twice by preparative HPLC (Gradient E). The fractions corresponding to the expected product were collected and then concentrated under reduced pressure to result in the compound GTP-gamma-N-octyl-thiosuccinimidyl-C2 (0.241 μmol, 24%) in the form of a colorless solution. LRMS (ESI+): calculated for C₅₁H₆₈EuN₁₃O₂₄P₃S₂⁺ [M+H]⁺, m/z=1555.8, found 1555.8.

### Binding Tests

### Materials

membrane preparations of cells expressing GPCRs and the G-alpha-i protein (Gαi) were purchased from Perkin Elmer or Euroscreen. The table below lists the cell backgrounds and references of the different samples used:

TABLE 1

| | Cell | | |
|---|---|---|---|
| | background | Provider | Reference |
| Delta Opioid | HEK293 | Perkin Elmer | 6110549400UA |
| Delta Opioid | CHO-K1 | Euroscreen | Service |
| Dopamine D2S | CHO-K1 | Euroscreen | Service |

the DSV36S antibody was generated by Cisbio Bioassays and is available from Cisbio Bioassays on request (under the reference DSV36S). The antibody was labeled with the fluorescent probe d2 (red acceptor) compatible for TR-FRET detection. The DSV36S antibody binds at the switch II of the Gαi protein.
the GTP and GTPγS nucleotides were purchased from Sigma Aldrich (respective catalog references G8877 and G8634).
the 384-well, low volume, white plates with a white background were purchased from Greiner Bio One (catalog reference 784075).
the non-hydrolyzable/slowly hydrolyzable GTP analogs labeled with donor fluorophores of lanthanide complex type were synthesized at Cisbio Bioassays.

### Methods

### Preparation of the Reagents

Except for other specific mention, all the reagents were diluted in 50 mM Tris HCl PH 7.4, 10 mM MgCl₂, 0.1% BSA, 10 mM NaCl, buffer. The membranes were prepared 4× to distribute 1 or 10 μg/well (amount specified in the key of each figure). The GTPyS nucleotide (non-specific signal condition) was prepared 6.67× to obtain a final concentration in the wells of 100 μM. The anti-Gαi antibody DSV36S-d2 used for the detection was prepared 4× to target the final concentration in the wells of 10 nM. The non-hydrolyzable/slowly hydrolyzable GTP analogs labeled with fluorescent donor probes (lanthanide complexes) were prepared 4× to target the final concentrations in the wells mentioned in the keys of each figure.

### Distribution of the Reagents in the 384-Well Plates:

Membranes expressing GPCR and the G protein: 5 μl
GTPγS buffer or nucleotide (for the non-specific signal condition): 3 μl
Non-hydrolyzable/slowly hydrolyzable GTP analog-donor: 5 μl
Anti-Gαi antibody-acceptor (DSV36S-d2): 5 μl
Buffer: 2 μl

The non-specific signal (background fluorescence noise) was measured with wells containing an excess of GTPγS (10 or 100 μM).

### Reading the HTRF Signal

The plates were incubated at 21° C. for 20 h and then the HTRF signal was measured on the PHERAstar reader (BMG Labtech) with the following configuration:
Module: HTRF (Excitation 337 nm, Emission 665 nm and 620 nm)
Excitation: laser, 40 flashes or lamp, 100 flashes
Reading window: delays: 60 μs-Integration: 400 μs

### Signal Processing

From the raw signals at 665 nm and 620 nm, the HTRF Ratio was calculated according to the following formula:
HTRF Ratio=[(Signal at 665 nm)/(Signal at 620 nm)]×10 000

### Test Format

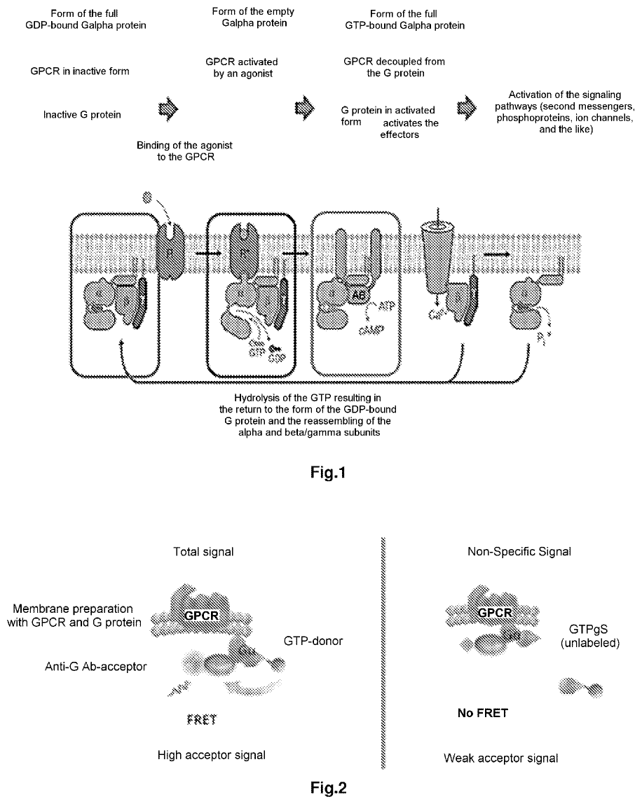

FIG. 2 illustrates the test format used to detect the binding of the compounds of formula (I) to the G protein. A membrane preparation comprising GPCRs and Gαi proteins was incubated in the presence of the test compounds and of an anti-Gα protein antibody labeled with an acceptor fluorophore, in the presence or absence of a non-hydrolyzable or slowly hydrolyzable GTP analog (typically, GTPγS). When the two FRET partners (test compound and anti-Gα protein antibody) bind to the same Gα protein, a FRET signal appears (left part of the figure: “Total Signal”) and is distinguished from the non-specific signal (background noise corresponding to the right part of the figure: “Non-Specific Signal”), which comprises a large excess of unlabeled GTPyS (displacement of the fluorescent GTP analog).

### Example 1—GTPgN-C2 Analog Binding Test

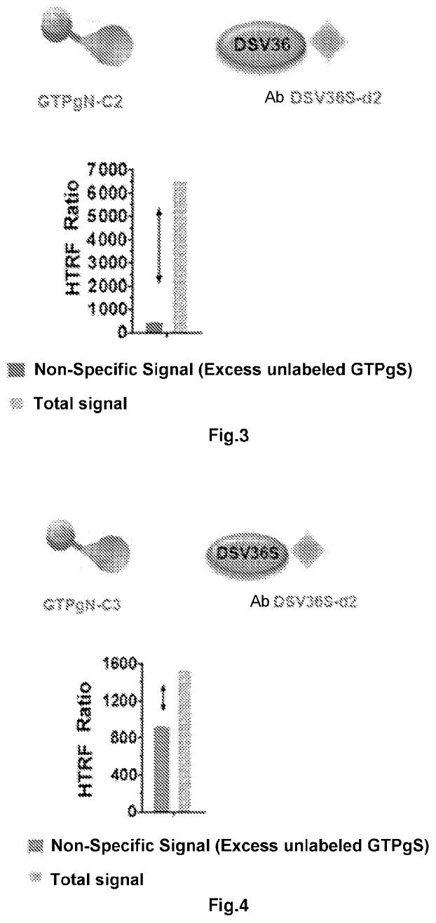

The ability of the GTPgN-C2 (preparation 2)/anti-Gαi antibody DSV36S-d2 pair to generate a specific TR-FRET signal by binding to the G protein was demonstrated using membrane preparations of CHO-K1 cells expressing the Delta Opioid GPCR and the Gαi protein (10 μg per well). GTPgN-C2 was used at a final concentration of 6 nM in the wells. The membranes were incubated in the absence or in the presence of a large excess of GTPγS (100 μM). The difference in TR-FRET signal (HTRF Ratio total signal/non-specific signal=16.6) observed between these two conditions shows that the GTPgN-C2 analog is capable of binding to the Gαi protein and generating a TR-FRET signal with an anti-Gαi antibody—acceptor (FIG. 3).

### Example 2—GTPgN-C3 Analog Binding Test

The ability of the GTPgN-C3 (preparation 5)/anti-Gαi antibody DSV36S-d2 pair to generate a specific TR-FRET signal by binding to the G protein was demonstrated using membrane preparations of HEK293 cells expressing the Delta Opioid GPCR and the Gαi protein (1 μg per well). GTPgN-C3 was used at a final concentration of 1 nM in the wells. The membranes were incubated in the absence or in the presence of a large excess of GTPγS (100 μM). The difference in TR-FRET signal (HTRF Ratio total signal/non-specific signal=1.7) observed between these two conditions shows that the GTPgN-C3 analog is capable of binding to the Gαi protein and generating a TR-FRET signal with an anti-Gα antibody-acceptor (FIG. 4).

### Example 3—GTPgN-Octyl-C2 Analog Binding Test

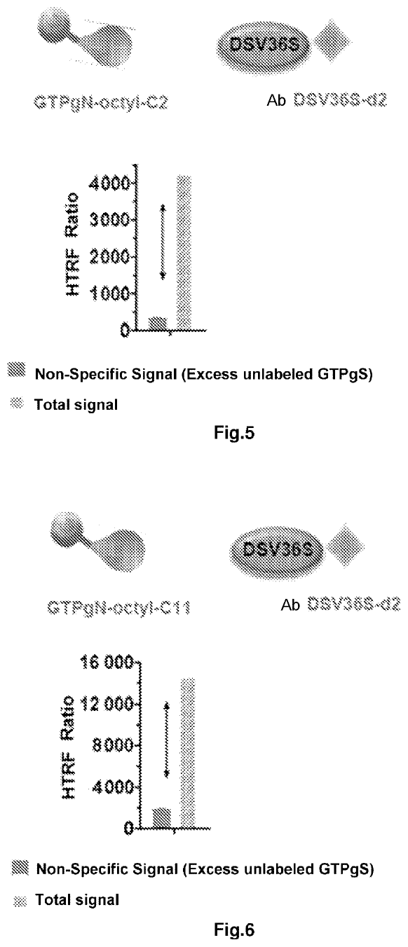

The ability of the GTPgN-octyl-C2 (preparation 6)/anti-Gαi antibody DSV36S-d2 pair to generate a specific TR-FRET signal by binding to the G protein was demonstrated using membrane preparations of CHO-K1 cells expressing the Delta Opioid GPCR and the Gαi protein (10 μg per well). GTPgN-octyl-C2 was used at a final concentration of 6 nM in the wells. The membranes were incubated in the absence or in the presence of a large excess of GTPγS (100 μM). The difference in TR-FRET signal (HTRF Ratio total signal/non-specific signal=12.4) observed between these two conditions shows that the GTPgN-octyl-C2 analog is capable of binding to the Gαi protein and generating a TR-FRET signal with an anti-Gαi antibody-acceptor (FIG. 5).

### Example 4—GTPgN-Octyl-C11 Analog Binding Test

The ability of the GTPgN-octyl-C11 (preparation 9)/anti-Gαi antibody DSV36S-d2 pair to generate a specific TR-FRET signal by binding to the G protein was demonstrated using membrane preparations of CHO-K1 cells expressing the Delta Opioid GPCR and the Gαi protein (10 μg per well). GTPgN-octyl-C11 was used at a final concentration of 6 nM in the wells. The membranes were incubated in the absence or in the presence of a large excess of GTPyS (100 μM). The difference in TR-FRET signal (HTRF Ratio total signal/non-specific signal=7.6) observed between these two conditions shows that the GTPgN-octyl-C11 analog is capable of binding to the Gαi protein and generating a TR-FRET signal with an anti-Gαi antibody-acceptor (FIG. 6).

### Example 5—GTPgN-Octyl-C3 Analog Binding Test

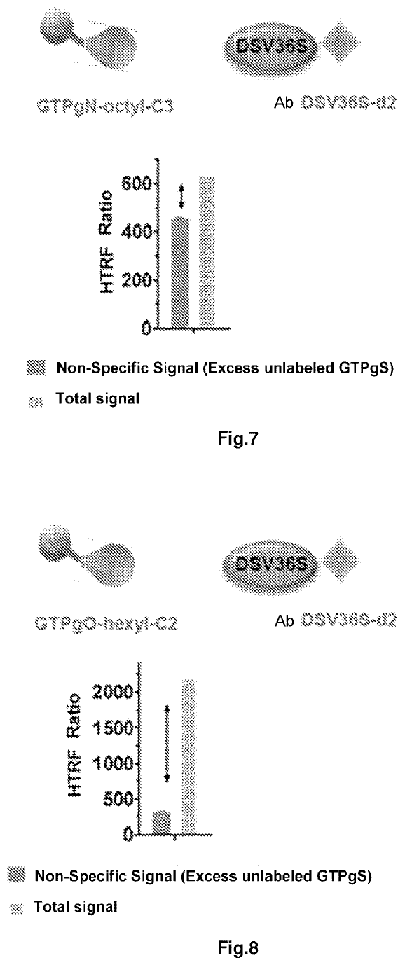

The ability of the GTPgN-octyl-C3 (preparation 7)/anti-Gαi antibody DSV36S-d2 pair to generate a specific TR-FRET signal by binding to the G protein was demonstrated using membrane preparations of HEK293 cells expressing the Delta Opioid GPCR and the Gαi protein (1 μg per well). GTPgN-octyl-C3 was used at a final concentration of 1 nM in the wells. The membranes were incubated in the absence or in the presence of a large excess of GTPγS (100 μM). The difference in TR-FRET signal (HTRF Ratio total signal/non-specific signal=1.4) observed between these two conditions shows that the GTPgN-octyl-C3 analog is capable of binding to the Gαi protein and generating a TR-FRET signal with an anti-Gαi antibody-acceptor (FIG. 7).

### Example 6—GTPgO-Hexyl-C2 Analog Binding Test

The ability of the GTPgO-hexyl-C2 (preparation 2)/anti-Gαi antibody DSV36S-d2 pair to generate a specific TR-FRET signal by binding to the G protein was demonstrated using membrane preparations of CHO-K1 cells expressing the Delta Opioid GPCR and the Gαi protein (10 μg per well). GTPgO-hexyl-C2 was used at a final concentration of 6 nM in the wells. The membranes were incubated in the absence or in the presence of a large excess of GTPγS (100 μM). The difference in TR-FRET signal (HTRF Ratio total signal/non-specific signal=7.1) observed between these two conditions shows that the GTPgO-hexyl-C2 analog is capable of binding to the Gαi protein and generating a TR-FRET signal with an anti-Gαi antibody-acceptor (FIG. 8).

### Example 7—GTPgO-Hexyl-C3 Analog Binding Test

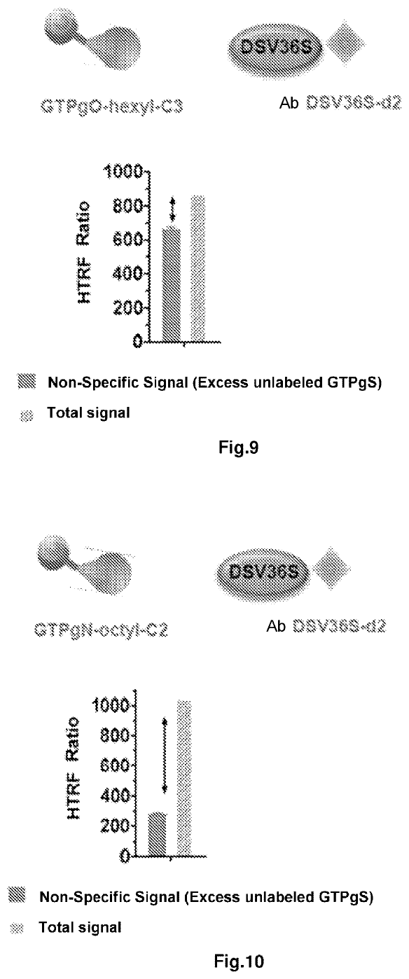

The ability of the GTPgO-hexyl-C3 (preparation 3)/anti-Gαi antibody DSV36S-d2 pair to generate a specific TR-FRET signal by binding to the G protein was demonstrated using membrane preparations of HEK293 cells expressing the Delta Opioid GPCR and the Gαi protein (1 μg per well). GTPgO-hexyl-C3 was used at a final concentration of 1 nM in the wells. The membranes were incubated in the absence or in the presence of a large excess of GTPγS (100 μM). The difference in TR-FRET signal (HTRF Ratio total signal/non-specific signal=1.3) observed between these two conditions shows that the GTPgO-hexyl-C3 analog is capable of binding to the Gαi protein and generating a TR-FRET signal with an anti-Gαi antibody-acceptor (FIG. 9).

### Example 8—GTPgN-Octyl-C2 Analog Binding Test

The ability of the GTPgN-octyl-C2 (preparation 6)/anti-Gαi antibody DSV36S-d2 pair to generate a specific TR-FRET signal by binding to the G protein was demonstrated using membrane preparations of CHO-K1 cells expressing the Dopamine D2S GPCR and the Gαi protein (10 μg per well). GTPgN-octyl-C2 was used at a final concentration of 6 nM in the wells. The membranes were incubated in the absence or in the presence of a large excess of GTPyS (100 μM). The difference in TR-FRET signal (HTRF Ratio total signal/non-specific signal=3.8) observed between these two conditions shows that the GTPgN-octyl-C2 analog is capable of binding to the Gαi protein and generating a TR-FRET signal with an anti-Gαi antibody-acceptor (FIG. 10).

### Example 9—Effect of the Concentration of Membranes and of GTP Analog-Lanthanide on the GTPgN-Octyl-C2 Analog Binding Test

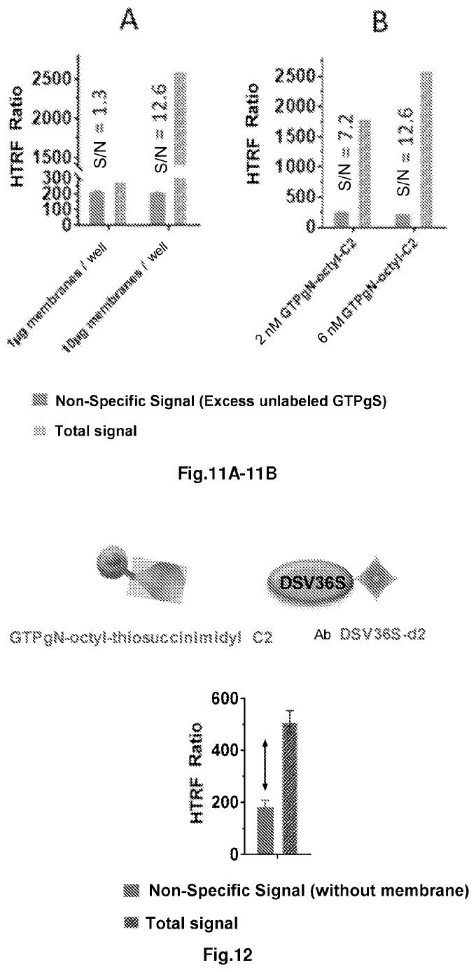

The ability of the GTPgN-octyl-C2 (preparation 6)/anti-Gαi antibody DSV36S-d2 pair to generate a specific TR-FRET signal by binding to the G protein was demonstrated using membrane preparations of CHO-K1 cells expressing the Delta Opioid GPCR and the Gαi protein (1 or 10 μg/well). GTPgN-octyl-C2 was used at a final concentration of 2 or 6 nM in the wells. The membranes were incubated in the absence or in the presence of a large excess of GTPγS (100 μM). The difference in TR-FRET signal (HTRF Ratio total signal(S)/non-specific signal (N)) observed between these two conditions shows that the GTPgN-octyl-C2 analog is capable of binding to the Gαi protein and generating a TR-FRET signal with an anti-Gαi antibody-acceptor (FIGS. 11A and 11B). Moreover, it emerges from FIG. 11A that an increase in the signal amplitude (S/N) is observed by increasing the amount of membrane from 1 to 10 μg per well. It emerges from FIG. 11B that an increase in the amplitude of the signal (S/N) is observed by increasing the concentration of GTPgN-octyl-C2 from 2 to 6 nM.

### Example 10—GTPgN-Octyl-Thiosuccinimidyl-C2 Analog Binding Test

The ability of the GTPgN-octyl-thiosuccinimidyl-C2 (preparation 10)/anti-Gαi antibody DSV36S-d2 pair to generate a specific TR-FRET signal by binding to the G protein was demonstrated using membrane preparations of CHO-K1 cells expressing the Delta Opioid GPCR and the Gαi protein (10 μg per well). The reagents were diluted in 50 mM Tris HCl buffer pH 7.4, 60 mM MgCl₂, 0.1% BSA, 150 mM NaCl. GTPgN-octyl-thiosuccinimidyl-C2 was used at a final concentration of 7.5 nM in the wells. The fluorescent conjugates were incubated in the absence or in the presence of membrane preparation. The difference in TR-FRET signal (HTRF Ratio total signal/non-specific signal=2.8) observed between these two conditions shows that the GTPgN-octyl-thiosuccinimidyl-C2 analog is capable of binding to the Gαi protein and generating a TR-FRET signal with an anti-Gαi antibody-acceptor (FIG. 12).

1. A compound of formula (I): 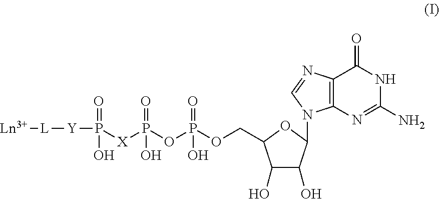
in which:
X=O;
Y=O, NH or CH₂;
L is a divalent linking group chosen from
a direct link; or
a linear or branched C₁-C₈ alkylene group optionally containing one or more oxygen atoms;
Ln³⁺ is a residue of a lanthanide complex which optionally carries a reactive group G₃;
G₃ is selected from the group consisting of: a succinimidyl ester, a halotriazine, and an isothiocyanate;
The lanthanide complex is selected from:

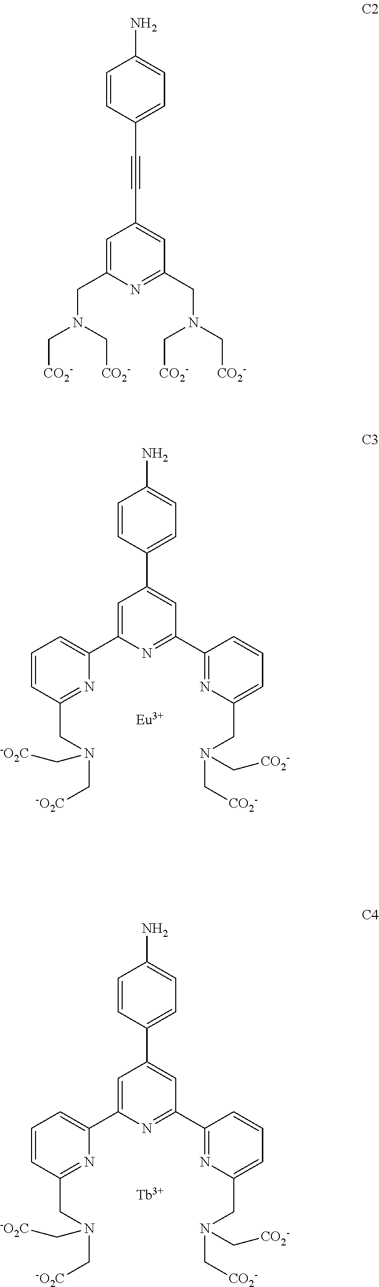

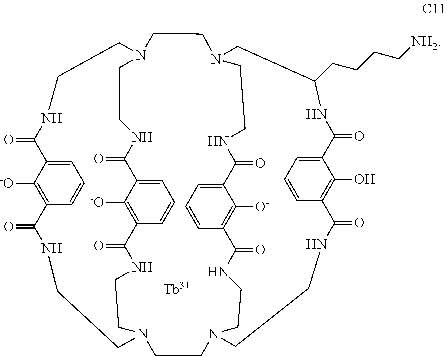

2. The compound of claim 1, wherein Y=O.

3. The compound of claim 1, wherein Y=NH.

4. The compound of claim 1, wherein Y=CH₂.

5. The compound of claim 1, wherein L is selected from the group consisting of: 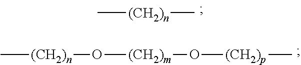
in which n, m, p are integers from 1 to 5.

6. The compound of claim 1, which is selected from the group consisting of:

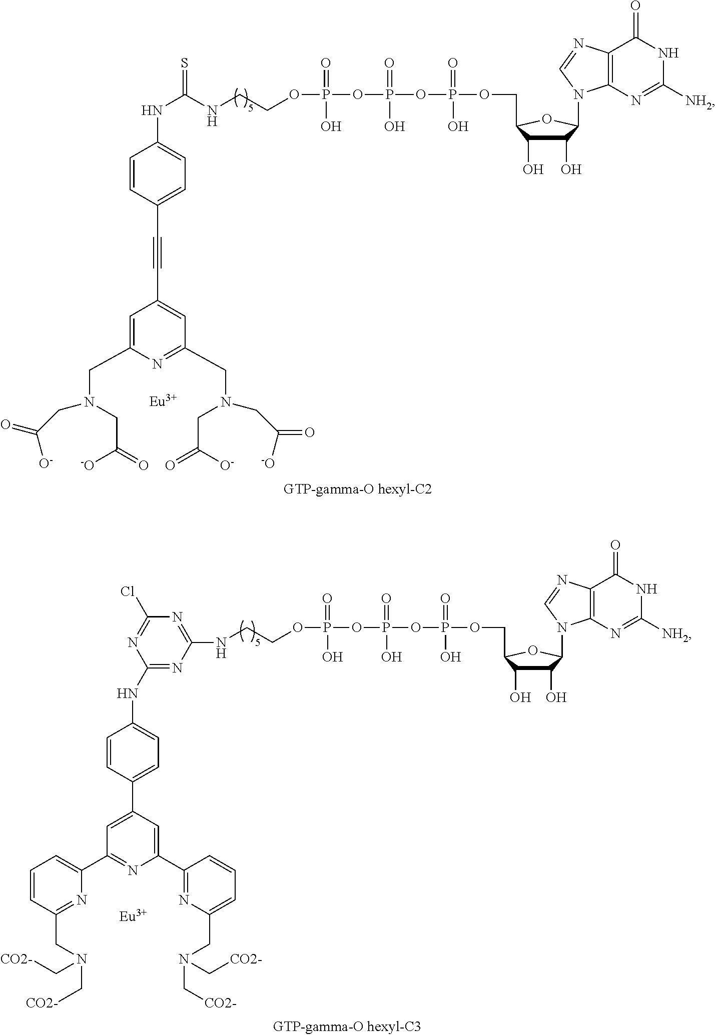

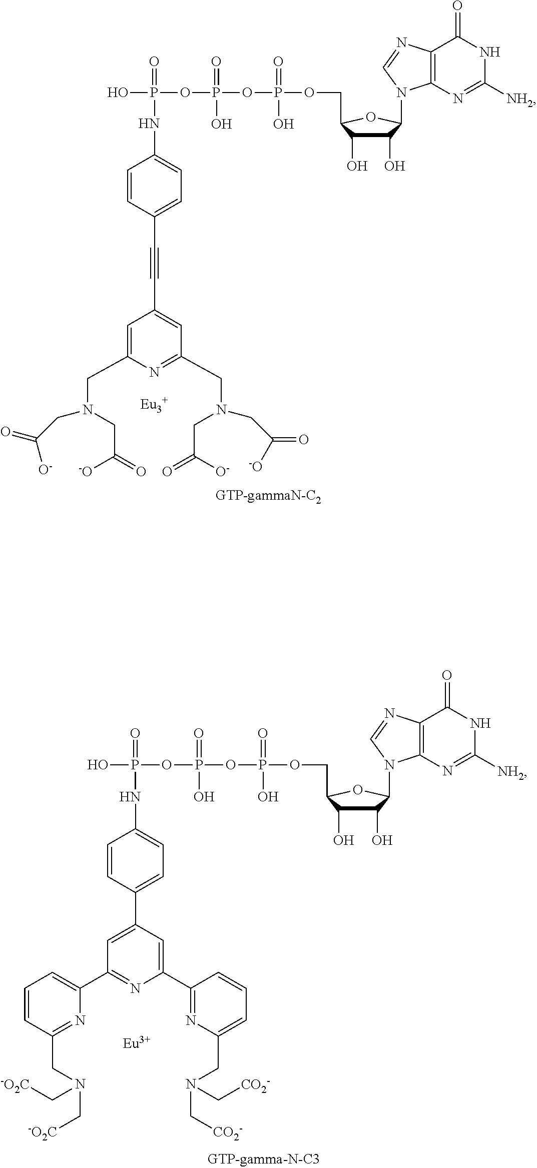

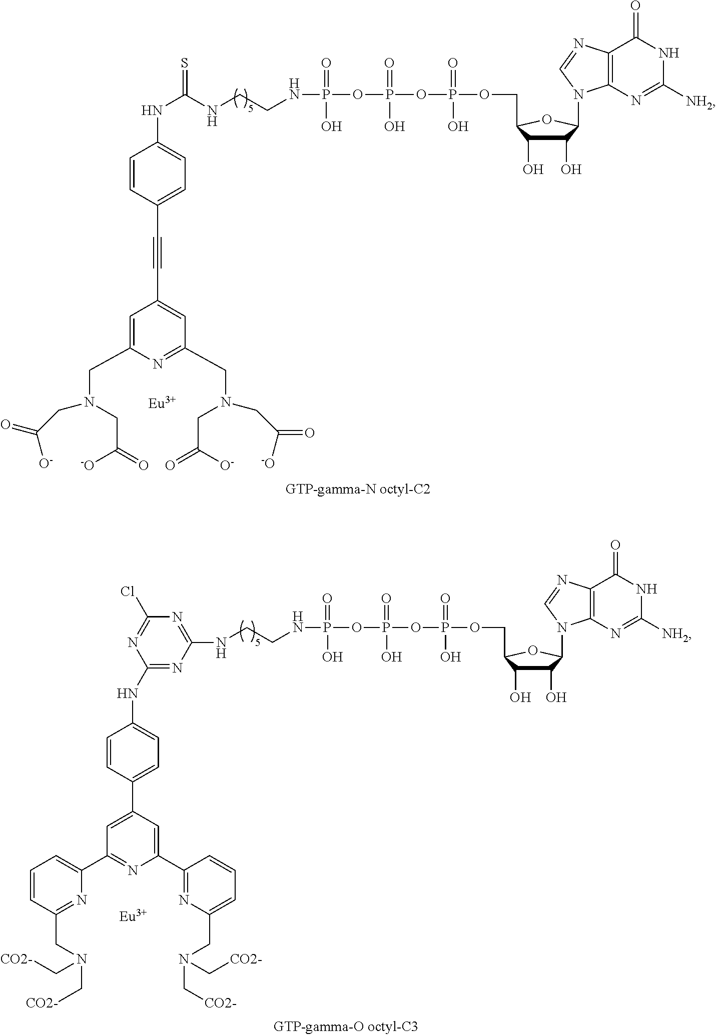

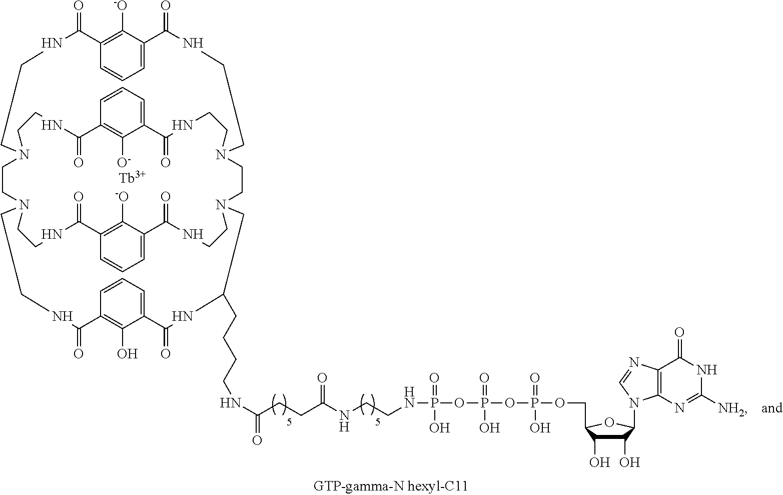

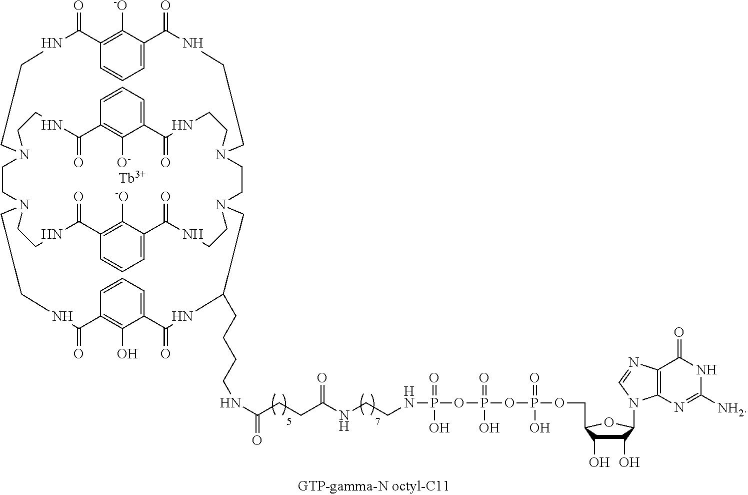

7. The compound of claim 6, which is selected from the group consisting of: [chemical structure]
